(19) United States
(12) Patent Application Publication
(10) Pub. No.: US 2025/0170153 A1
(43) Pub. Date: May 29, 2025

(54) METHODS FOR THE TREATMENT OF ANXIETY DISORDER

(71) Applicants: ANANDA SCIENTIFIC, INC., Greenwood Village, CO (US); LYOTROPIC DELIVERY SYSTEMS LTD., Jerusalem (IL)

(72) Inventors: Mark J. ROSENFELD, Greenwood Village, CO (US); Sohail R. ZAIDI, Greenwood Village, CO (US); Christopher B.G. MOORE, Greenwood Village, CO (US)

(21) Appl. No.: 18/859,589

(22) PCT Filed: May 5, 2023
(86) PCT No.: PCT/US2023/021216

§ 371 Date: Oct. 24, 2024

Related U.S. Application Data
(60) Provisional application No. 63/338,777, filed on May 5, 2022.

(51) Int. Cl. A61K 31/00; A61K 9/00; A61K 9/48; A61P 25/22

(52) U.S. Cl. CPC A61K 31/658; A61K 9/4825; A61K 9/4858; A61K 9/4866; A61K 9/4875; A61P 25/22; A61K 9/0053

(57) ABSTRACT

The present disclosure is directed to methods of effectively treating or preventing anxiety disorders, e.g., social anxiety disorder, with pharmaceutical compositions comprising at least one cannabinoid, as well as methods of making and kits containing said compositions. Finally, methods of predicting a subject's probability of response and selecting subjects most likely to benefit from CBD based treatment are disclosed.

### FIELD OF THE DISCLOSURE

The present disclosure presents compositions comprising one or more cannabinoids (e.g., cannabidiol). The present disclosure presents compositions comprising one or more cannabinoids (e.g., cannabidiol) for use in the treatment of diseases and disorders, including anxiety disorder, e.g., generalized anxiety disorder, panic disorder, social anxiety disorder, phobia-related disorder (e.g., aerophobia, acrophobia, zoophobia, trypanophobia, hemophobia, agoraphobia, or the like), separation anxiety disorder, selective mutism, medication-induced anxiety disorder, or the like.

### BACKGROUND OF THE DISCLOSURE

Cannabidiol (CBD) is a promising candidate for treating a range of diseases and disorders, including anxiety disorder, e.g., generalized anxiety disorder, panic disorder, social anxiety disorder, phobia-related disorder (e.g., aerophobia, acrophobia, zoophobia, trypanophobia, hemophobia, agoraphobia, or the like), separation anxiety disorder, selective mutism, medication-induced anxiety disorder, or the like, as well as associated symptoms and neurocognitive impairments. CBD has been shown to mitigate key neurocircuit disturbances underlying these disorders, symptoms, and cognitive impairments. Extensive evidence in animal models also shows that CBD reduces anxiety behaviors, compulsive behaviors, panic response, and physiological stress responses by inhibiting extended amygdala activation and promoting response in the hippocampus and medial prefrontal cortex. Cannabidiol also modifies consolidation and reconsolidation, suggesting the ability to modify traumatic memories. CBD's anxiolytic and pro-extinction effects depend on inhibition of extended amygdala activity and facilitation of PFC and HPC activity. Though the mechanism of action of CBD is not fully understood, CBD has been shown to have an inhibitory effect at CB1 and CB2 receptors and to correspondingly alter the “bias” of systems activated by CB1 agonists (i.e., endocannabinoids and THC). Like other non-psychoactive cannabinoids, CBD shows low affinity for CB1 and CB2 receptors, and is not an orthosteric ligand at those sites.

Historically, cannabinoids have been used anecdotally as a treatment for a wide array of conditions. However, more recent scientific research has begun to explore these compounds for reliable medicinal use. To date, the focus of this research has been predominantly on two compounds as potential therapeutics: tetrahydrocannabinol (THC) and cannabidiol (CBD). THC is a psychoactive compound, presenting long lasting adverse side effects on the user. CBD, however, is non-psychoactive and is considered non-intoxicating and safe for various routes of administration. THC and CBD are most commonly found together as a mixture in various concentrations in plant sources, and as a result, most therapeutic applications currently known involve consumption of both compounds together. A need therefore exists for formulations and pharmaceutical compositions which comprise CBD and are free of psychoactive cannabinoids such as THC, thereby minimizing the deleterious side effects of THC, such as intoxication.

As a class, cannabinoids are non-water soluble. This has posed a challenge both in the extraction of cannabinoids from natural sources and in formulating pharmaceutical compositions for oral administration. Cannabinoids are lipid soluble, and CBD has been delivered orally in oil-based capsules in human trials. However, due to CBD's low water solubility, absorption from the gastrointestinal system is erratic and leads to variable pharmacokinetics (see, e.g., Taylor, L., et al., CNS drugs, 2018 November; 32 (11): 1053-67, and Millar, S. A., et al., Frontiers in pharmacology. 2018 Nov. 26; 9:1365, the contents of which are each incorporated herein by reference in their entireties, as related to the preparation and use of CBD formulations). Bioavailability of cannabinoids administered orally is generally low (less than 10% in some reports), largely dose dependent, and variable. A need therefore exists for reliable pharmaceutical compositions containing water soluble cannabinoid compositions which can be effectively delivered orally, and which improve the overall bioavailability of orally administered cannabinoids.

### SUMMARY OF THE DISCLOSURE

The present disclosure presents methods of treating an anxiety disorder, e.g., generalized anxiety disorder, panic disorder, social anxiety disorder, phobia-related disorder (e.g., aerophobia, acrophobia, zoophobia, trypanophobia, hemophobia, agoraphobia, or the like), separation anxiety disorder, selective mutism, medication-induced anxiety disorder, or the like, in a subject in need thereof comprising administering to a subject a therapeutically effective amount of a pharmaceutical composition comprising at least one cannabinoid, at least one oil, at least one hydrophilic surfactant, at least one co-surfactant, less than 1 wt % water, optionally, at least one solvent, optionally, at least one co-solvent optionally, at least one phospholipid, and optionally, at least one additive. In some embodiments the anxiety disorder is social anxiety disorder generalized anxiety disorder, panic disorder, phobia-related disorder, separation anxiety disorder, selective mutism, or medication-induced anxiety disorder.

In some embodiments, the method includes reducing a Structured Clinical Interview Schedule for DSM-5 (SCID-5) for social anxiety disorder by the administration of the pharmaceutical composition to the subject, as compared to the subject's pre-treatment score.

In some embodiments, the method includes reducing a Trier Social Stress Test (TSST) score for social anxiety by the administration of the pharmaceutical composition to the subject, as compared to the subject's pre-treatment score.

In some embodiments, the method includes reducing a Liebowitz Social Anxiety Scale (LSAS) score for social anxiety by the administration of the pharmaceutical composition to the subject in need thereof, as compared to the subject's pre-treatment score.

In some embodiments, the method includes reducing a Clinical Global Impression of Severity and Improvement (CGI-I and S) score by the administration of the pharmaceutical composition to the subject in need thereof, as compared to the subject's pre-treatment score.

In some embodiments, the method includes reducing a General Anxiety Disorder (GAD-7) score in the subject by the administration of the pharmaceutical composition to the subject, as compared to the subject's pre-treatment score.

In some embodiments, the method includes reducing a Quick Inventory of Depressive Symptomology, Self-Report (QIDS-SR) score in the subject by the administration of the pharmaceutical composition to the subject, as compared to the subject's pre-treatment score.

In some embodiments, the method includes reducing a Perceived Stress Scale (PSS-14) score in the subject by the administration of the pharmaceutical composition to the subject, as compared to the subject's pre-treatment score.

In some embodiments, the method includes reducing an Anxiety Sensitivity Index-3 (ASI-3) score in the subject by the administration of the pharmaceutical composition to the subject, as compared to the subject's pre-treatment score.

In some embodiments, the method includes reducing an Insomnia Severity Index score in the subject by the administration of the pharmaceutical composition to the subject, as compared to the subject's pre-treatment score.

In some embodiments, the method includes reducing a PROMIS Anxiety score in the subject by the administration of the pharmaceutical composition to the subject, as compared to the subject's pre-treatment score.

In some embodiments, the method includes reducing an Altman Self-Rating Mania Scale (ASRM) score in the subject by the administration of the pharmaceutical composition to the subject, as compared to the subject's pre-treatment score.

In some embodiments, the method includes reducing a PROMIS Emotional Distress score in the subject by the administration of the pharmaceutical composition to the subject, as compared to the subject's pre-treatment score.

In some embodiments, the method includes reducing a Patient Health Questionnaire 15 Somatic Symptom Severity Scale (PHQ-15) score in the subject by the administration of the pharmaceutical composition to the subject, as compared to the subject's pre-treatment score.

In some embodiments, the method includes reducing a PROMIS Sleep Disturbance score in the subject by the administration of the pharmaceutical composition to the subject, as compared to the subject's pre-treatment score.

In some embodiments, the method includes reducing a Florida Obsessive-Compulsive Inventory (FOCI) Severity Scale (Part B) score in the subject by the administration of the pharmaceutical composition to the subject, as compared to the subject's pre-treatment score.

In some embodiments, the method includes reducing a Substance Use score in the subject by the administration of the pharmaceutical composition to the subject, as compared to the subject's pre-treatment score.

In some embodiments, the method includes reducing a Severity Measure for Depression, e.g., Patient Health Questionnaire (PHQ-9), score in the subject by the administration of the pharmaceutical composition to the subject, as compared to the subject's pre-treatment score.

In some embodiments, the method includes reducing a Severity Measure for Separation Anxiety Disorder score in the subject by the administration of the pharmaceutical composition to the subject, as compared to the subject's pre-treatment score.

In some embodiments, the method includes reducing a Severity Measure for Specific Phobia score in the subject by the administration of the pharmaceutical composition to the subject, as compared to the subject's pre-treatment score.

In some embodiments, the method includes reducing a Severity Measure for Panic Disorder score in the subject by the administration of the pharmaceutical composition to the subject, as compared to the subject's pre-treatment score.

In some embodiments, the method includes reducing a Severity Measure for Agoraphobia score in the subject by the administration of the pharmaceutical composition to the subject, as compared to the subject's pre-treatment score.

In some embodiments, the method includes reducing a Severity of Dissociative Symptoms, e.g., Brief Dissociative Experiences Scale (DES-B), score in the subject by the administration of the pharmaceutical composition to the subject, as compared to the subject's pre-treatment score.

In some embodiments, the total daily dose of cannabinoid administered to the subject is at least 50 mg/day. In some embodiments, the total daily dose of cannabinoid administered to the subject is from 50 mg/day to 100 mg/day. In some embodiments, the total daily dose of cannabinoid administered to the subject is from 100 mg/day to 2000 mg/day. In some embodiments, the total daily dose of cannabinoid administered to the subject is from 200 mg/day to 1400 mg/day. In some embodiments, the total daily dose of cannabinoid administered to the subject is from 200 mg/day to 600 mg/day. In some embodiments, the total daily dose of cannabinoid administered to the subject is from 700 mg/day to 1400 mg/day. In some embodiments, the total daily dose of cannabinoid administered to the subject is 200 mg/day, 400 mg/day, 600 mg/day, 700 mg/day, 1400 mg/day, or 2000 mg/day.

In some embodiments, the total daily dose of cannabinoids is administered to the subject in a single daily dose.

In some embodiments, the total daily dose of cannabinoids is administered to the subject as a split daily dose. In some embodiments, the total daily dose of cannabinoids is administered to the subject as a split daily dose comprising 2, 3, or 4 smaller doses. In some embodiments, the split daily dose comprises smaller doses having substantially equivalent cannabinoid concentration. In some embodiments, the split daily dose comprises smaller doses having inequivalent cannabinoid concentration.

In some embodiments, the pharmaceutical composition is administered via at least one of route selected from topical, buccal, sublingual, dental, oral, otic, rectal, vaginal, endocervical, transdermal, subcutaneous, intravenous, intramuscular, transdermal, intranasal, inhalation, ocularly, gavage, or parenterally into the circulatory system of a subject.

In some embodiments, the pharmaceutical composition is administered in at least one form selected from a tablet, a gel, a powder, a lotion, an oil, a soap, a spray, an emulsion, a cream, an ointment, a capsule, soft gel capsules, chewing gum, a patch, a buccal-patch a nutraceutical a dietary supplement, or a solution.

In some embodiments, the pharmaceutical composition is in the form of a soft gel capsule for administration orally as a split daily dose and provides a total daily dose of CBD of 600 mg/day to 2000 mg/day.

In some embodiments, the pharmaceutical composition is in the form of a soft gel capsule for administration orally as a split daily dose and provides a total daily dose of CBD of 400 mg/day.

In some embodiments, the total daily dose of CBD changes over the course of treatment, e.g., the total daily dose of CBD increases from 200 mg/day CBD to 400 mg/day CBD after 1 week of treatment, e.g., the total daily dose of CBD increases from 200 mg/day CBD to 400 mg/day CBD after 2 weeks of treatment, e.g., the total daily dose of CBD increases from 400 mg/day CBD to 600 mg/day CBD after 2 weeks of treatment.

In another aspect, the disclosure presents a kit comprising the pharmaceutical composition and instructions for the use of said composition, in which the kit is for the treatment of an anxiety disorder.

In another aspect, the disclosure presents a method of producing a Probability of Response (POR) of a subject to any of the methods of treating anxiety disorder recited in the preceding claims, which includes collecting a subject's demographic, clinical (including medication and substance use information), and biological (i.e., genetic, and other blood-based markers) information as well as task-based and structural magnetic resonance neuroimaging markers centered around hippocampal, prefrontal, and extended amygdala function and fear extinction before administration of the pharmaceutical composition or placebo, administering the pharmaceutical composition or placebo and collecting the measures of again after administration, and applying a random forest algorithm to generate a POR.

In another aspect, the disclosure presents a method for selecting subjects most likely to benefit from any of the methods of treating anxiety disorder recited in the preceding claims, which includes collecting a subject's demographic, clinical (including medication and substance use information), and biological (i.e., genetic, and other blood-based markers) information as well as task-based and structural magnetic resonance neuroimaging markers centered around hippocampal, prefrontal, and extended amygdala function and fear extinction, applying a probability classifier to the features collected to predict the POR to the pharmaceutical composition for the subject, and selecting subjects who have a predicted POR greater than a determined threshold (e.g., POR >0.6).

### Definitions

As used herein, the terms “active ingredient”, “therapeutic agent”, and “therapeutic ingredient” refer to the component of a pharmaceutical composition which is biologically active, such as a cannabinoid.

As used herein, the term “administer” and its grammatical equivalents refer to providing a formulation or pharmaceutical composition to a subject. Administration can include continuous administration or intermittent administration.

As used herein, the term “adjuvants” refers to any substance or a combination of substances, that is used to increase the efficacy or potency of another drug.

As used herein, the terms “approximately” and “about,” as applied to one or more values of interest, refer to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, “between” two numbers is intended to include the two numbers themselves. Accordingly, “between 0.5 and 20 wt %” include 0.5 wt %, 20 wt %, and all wt % from 0.5 and 20 wt %.

As used herein, the term “bioavailability” refers to the proportion of a drug or other substance which enters systemic circulation when introduced into the body of a subject.

As used herein, the terms “cannabidiol” and “CBD” refer to a non-psychoactive cannabinoid of the same name, having a chemical formula C₂₁H₃₀O₂ and an IUPAC name 2-(1R,6R)-3-methyl-6-prop-1-en-2-ylcyclohex-2-en-1-yl-5-pentylbenzene-1,3-diol (See FIG. 2). The term also encompasses derivative compounds which are the product of chemical modification to a naturally occurring cannabidiol, so long as the product retains its therapeutic activity.

As used herein, the term “cannabinoid” refers to therapeutically active compounds which are found in plants of the genus Cannabis (e.g., Cannabis sativa, a.k.a., hemp). The term includes compounds which are obtained from natural sources (e.g., plants), as well as compounds obtained synthetically. The term also encompasses derivative compounds which are the product of chemical modification to a naturally occurring cannabinoid, so long as the product retains its therapeutic activity.

As used herein the term “cannabinoid source” refers to a source, (natural, semi-synthetic or synthetic) that contains a cannabinoid. Examples of cannabinoid sources include, but are not limited to, substantially pure cannabinoid (e.g., pure CBD), a cannabinoid in crystalline form, a natural cannabinoid source (e.g., cannabis plant or part thereof), and a cannabinoid extract (e.g., extract obtained by known extraction methods).

As used herein, the term “co-solvent” refers to a second solvent included in a formulation which differs from a first solvent also included in the formulation.

As used herein, the term “co-surfactant” refers to a second surfactant agent in a formulation which differs from a first surfactant in the formulation (e.g., hydrophilic surfactant). Co-surfactants can refer to a second surfactant which is capable (together with the first surfactant) of lowering the interfacial tension between an oil phase and an aqueous phase to almost zero (or zero), allowing for the formation of a homogeneous mixture once the formulation is mixed with an aqueous liquid.

As used herein, the terms “daily dose” and “total daily dose” refer to the total amount of active ingredient to be administered to a subject in a given 24-hour period.

As used herein, the term “diluent” refers to any substance capable of diluting a pharmaceutical composition.

As used herein, the term “excipients” refers to any substance included in a pharmaceutical composition other than the active ingredient.

As used herein, the term “formulation” refers to a mixture of components combined in defined proportions. A formulation may be, but is not limited to, any one of the following forms: a microemulsion (ME), a liquid nanodomain, a nano-emulsion (NE), a micelle, a reverse micelle, a lipid nanoparticle (LNP), a nanoparticle, a suspension, a solution, an emulsion, a solid lipid nanoparticle (SLNP), a liposome, a nanosphere, a composite, a mixture, a macro particle, or an aggregate. The terms formulation and composition may be used interchangeably herein.

The term “homogenization,” as used herein, refers to the process of applying sheer forces onto mixtures to form intimate contact that permits the solubilization of the desired cannabinoid from the source. Homogenization may be carried out by any suitable means, including, but not limited to homogenizers and high-speed mechanical stirring.

The term “hydrophilic surfactant,” as used herein, refers to ionic or non-ionic surfactants having a hydrophilic nature, i.e., a surfactant having an affinity for water.

The term “natural cannabinoid,” as used herein, refers to any cannabinoid obtained from a plant by various processes of treatment or extraction. The source organism may be, without limitation, a wild type (i.e., naturally occurring) strain, any horticultural variant, any cultivated strain, or any engineered (e.g., genetically modified) strain in which the cannabinoid of interest can be found.

The term “mixing,” as used herein, refers to any suitable known method for combining components that does not involve sheer-mixing, for example, manual mixing, magnetically stirring, mixing by pedals and others.

The term “non-psychoactive cannabinoid,” as used herein, refers to a class of cannabinoids that do not cause intoxicating effects, i.e., it lacks the psychotomimetic, mild-altering effects as seen in psychoactive cannabinoids.

As used herein, the term “pharmaceutically acceptable” refers to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to any excipient (e.g., vehicles, adjuvants, or dilutants) which are capable of suspending, dissolving, encapsulating, or otherwise carrying an active ingredient in a formulation. Pharmaceutically acceptable carriers can function to improve the selectivity, effectiveness, and/or safety of delivery of an active ingredient.

As used herein, the term “pharmaceutical composition” refers to a composition comprising at least one active ingredient (e.g., cannabinoid), and at least one pharmaceutically acceptable carrier (e.g., formulation mixture)

The term “psychoactive cannabinoid,” as used herein, refers to a class of cannabinoids that appear to cause intoxicating effects.

As used herein, “purify,” “purified,” and “purification” mean to make substantially pure or clear from unwanted components, material defilement, admixture, or imperfection. “Purified” refers to the state of being pure. “Purification” refers to the process of making pure.

The term “single daily dose,” as used herein, refers to administering the total amount of active ingredient (e.g., cannabinoid) indicated by the method of treatment for a day to a subject at the same time. For example, a 600 mg dose of CBD taken once a day is a single daily dose administration.

The term “split daily dose,” as used herein, refers to administering the total amount of active ingredient (cannabinoid) indicated by the method of treatment for a day to a subject over the course of the day. For example, a 600 mg dose of CBD taken as 300 mg of CBD in the morning and 300 mg of CBD at night is a split daily dose administration schedule.

As used herein, the terms “subject” and “patient” refer to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, may receive treatment, or is under care by a trained professional for a particular disease or condition.

The term “synthetic cannabinoid,” as used herein, refers to any cannabinoid obtained by chemical synthesis or modification procedures.

As used herein, the terms “therapeutically effective amount” and “effective amount” refer to any amount of an active ingredient that can cause the desired effect (e.g., clinical results) when administered to a subject. An effective amount may be determined according to considerations known in the art, and one skilled in the art will recognize that the effective amount can depend on a variety of factors including: the distribution profile within the body, a variety of pharmacological parameters (e.g., half-life in the body), undesired side effects (if any), factors such as age and gender, and other considerations.

As used herein, the terms “treatment,” “treating,” and their grammatical equivalents refer to partially or completely alleviating, ameliorating, improving, relieving, delaying onset of, inhibiting progression of, reducing severity of, and/or reducing incidence of one or more symptoms or features of a particular infection, disease, disorder, and/or condition. Examples of treatment can include, but are not limited to: to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, to prevent the disease from occurring, or a combination thereof. Treatment may be administered to a subject who does not exhibit signs of a disease, disorder, and/or condition, and/or to a subject who exhibits only early signs of a disease, disorder, and/or condition for the purpose of decreasing the risk of developing pathology associated with the disease, disorder, and/or condition.

The term “vehicle,” as used herein, refers to any substance combined with an active ingredient to facilitate administration.

As used herein, the term “water-free” refers to a formulation that contains less than 1 wt % of water (i.e., essentially zero water). Water-free formulations do not include any amount of water purposefully added as a component during their formation. Water-free formulations may contain, for example, 0, less than 0.000001 wt %, less than 0.00001 wt %, less than 0.0001 wt %, less than 0.001 wt %, less than 0.01 wt %, less than 0.1 wt %, or less than 1 wt % of water. Water-free formulations may be referred to as concentrated formulations or concentrates. Such concentrated formulations may later be diluted, in water or other liquids, as needed for the effective practice of the disclosed methods, or the amount of water in the formulation may increase beyond 1 wt % over time to hydration by atmospheric water.

### BRIEF DESCRIPTION OF THE DRAWINGS

A better understanding of the features and advantages presented herein will be obtained by reference to the following detailed description that sets forth illustrative embodiments, and the accompanying drawings of which:

FIG. 1 shows a diagram of liquid nanodomains loaded with cannabidiol (CBD) upon dilution with an aqueous phase. In a form of water in oil structure at low aqueous phase content (a), bicontinuous mesophase at intermediate aqueous phase content (b), and oil in water nanostructures at high aqueous phase content (c).

FIG. 2 shows the chemical structure (Lewis structure) of CBD.

### DETAILED DESCRIPTION OF THE DISCLOSURE

### Cannabinoids

The present disclosure presents compositions comprising at least one cannabinoid.

Cannabinoids for use in the present disclosure include, but are not limited to, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CECA), cannabichromene (CBC), cannabichromevarinic acid (CBCV A), cannabichromevarin (CBCV), cannabidiolic acid (CBDA), cannabidiol (CDB), cannabidiol monomethylether (CBDM), cannabidiol-C4 (CBD-C4), cannabidivarin (CBDV), cannabidivarinic acid (CBDV A), cannabidiorcol (CBD-C1), delta-9-tetrahydrocannabinolic acid A (THCAA), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THCA-C4), delta-9-tetrahydrocannabivarinic acid (THCVA), delta-9-tetrahydrocannabivarin (THCV), delta-9-tetrahydrocannabiorcolic acid (THCAC1), delta-9-tetrahydrocannabiorcol (THC-Ci), delta-7-cis-iso-tetrahydrocannabivarin, delta-8-tetrahydrocannabinolic acid A (118-THCA), delta-8-tetrahydrocannabinol (118-THC), cannabicyclolic acid (CBLA), cannabicyclol (CBL), cannabicyclovarin (CBL V), cannabielsoic acid A (CBEA-A), cannabielsoic acid B (CBEA-B), cannabielsoin (CBE), cannabinolic acid (CENA), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 1 0-ethoxy-9-hydroxy-delta-6a-tetrahydrocannabinol, 8,9-dihydroxy-delta-6a-tetrahydrocannabinol, cannabitriolvarin (CBTV), ethoxycannabitriolvarin (CBTVE), dehydrocannabifuran (DCBF), cannabifuran (CBF), cannabichromanon (CBCN), cannabicitran (CBT), 10-oxo-delta-6atetrahydrocannabinol (OTHC), delta-9-cis-tetrahydrocannabinol (cis-THC), 3,4,5,6-tetrahtdro-7-hydroxy-a-a-2-trimethyl-9-n-propyl-2,6-methano-2H-I-benzoxocin-5-methanol (OH-iso-HHCV), cannabiripsol (CBR), and trihydroxy-delta-9-tetrahydroxycannabinol (triOH-THC).

In some embodiments the cannabinoid is a non-psychoactive cannabinoid. Non-psychoactive cannabinoids can include, but are not limited to, CBD, CBG, CBC, and CBDV. In some embodiments, the non-psychoactive cannabinoid is CBD or a CBD derivative.

In some cases, the cannabinoid (e.g., CBD) is a natural cannabinoid, i.e., one obtained via extraction from or treatment of a cannabinoid producing organism (e.g., plant). Examples of extraction methods include, but are not limited to, extraction by carrier oils, extraction by organic solvents, and/or super-critical CO₂ extraction. In some embodiments, cannabinoid extraction may be carried out utilizing methods, techniques, and formulations as presented in US 2019/0231833, the content of which is incorporated herein by reference in its entirety as related to the extraction, formulation, and use of cannabinoids.

In some embodiments, the cannabinoid is extracted in a process comprised of selecting a plant or synthetic source which contains the cannabinoid desired; mixing the source containing the cannabinoid with appropriate oils, solvents, and/or carriers; and filtering the resultant mixture. In some embodiments, the cannabinoid is extracted in a process comprised of selecting a plant or synthetic source which contains the cannabinoid desired; optionally using techniques known in the art to break down plant cell walls; mixing the source containing the cannabinoid with appropriate oils, solvents, and/or carriers, optionally under super critical carbon dioxide conditions; filtering the resultant mixture; and optionally concentrating, and/or purifying the mixture.

In some embodiments, the cannabinoid is extracted from a plant. In some embodiments, the cannabinoid is extracted from a plant of the Cannabis genus. In some embodiments, the cannabinoid is extracted from a Cannabis sativa (hemp) plant. In some embodiments, the cannabinoid is synthesized from one or more chemical synthesis.

In some embodiments, the cannabinoid is a synthetic cannabinoid. In some embodiments, the cannabinoid is a synthetic cannabinoid obtained via chemical synthesis or modification techniques.

In certain embodiments, the cannabinoid can target one or more pharmacological targets. In certain embodiments, the cannabinoid can target one or more pharmacological targets in the endocannabinoid system, including targets that are relevant to potential anxiolytic and/or pro-cognitive effects of the cannabinoid. Receptors of the endocannabinoid system are highly expressed in brain regions involved in the regulation of behaviors affected by anxiety disorders, e.g., social anxiety disorder, including: the hippocampus, amygdala, striatum, nucleus accumbens, frontal cortex, and entorhinal cortex. In certain embodiments, the cannabinoid (e.g., CBD) can target pharmacological targets/receptors related to the medicinal treatment of anxiety disorders, e.g., social anxiety disorder, symptoms. In certain embodiments, the cannabinoid (e.g., CBD) can be used in the medicinal treatment of anxiety disorders, e.g., social anxiety disorder, symptoms.

In at least ten studies of CBD's subjective anxiolytic effects in humans, a range of doses were successful at reducing anxiety to various triggers. In neuroimaging studies, these effects were accompanied by changes in neural circuits underlying anxiety and sympathetic arousal measured by skin conductance. CBD was also found to enhance fear extinction in humans.

In rodents, CBD has shown a combination of properties that suggest it may be effective for treatment of anxiety disorders symptoms in humans. As seen in several animal models of neurodegenerative disease, CBD attenuates cognitive impairment, protects the hippocampus (HPC) against the effects of chronic stress and increases hippocampal neurogenesis, and improves cognition in rodent models.

The therapeutic effects in rodents have been attributed in part to CBD enhancing the endocannabinoid system function and reducing systemic inflammation. Both factors are associated with anxiety disorder, e.g., social anxiety disorder pathophysiology.

Despite the apparent potential of CBD for use as a therapeutic agent, it has proven difficult to make use of reliably. Traditional methods of administering cannabinoids predominantly involve inhalation of cannabinoid containing smoke or vapors. In addition to the inherent negative health effects inherent in smoke and vapor inhalation, when administered in such a manner dose amounts tend to be inaccurate and variable. Additionally, the pharmacokinetics of CBD administered via inhalation is too variable to allow for consistent and reliable therapeutic administration. To date, methods of oral administration have suffered from extremely poor absorption and bioavailability of CBD.

The disclosed formulations overcome these limitations by allowing for an oral administration of CBD with increased bioavailability.

In some embodiments, the at least one cannabinoid comprises a CBD derivative (e.g., metabolite). In some embodiments, the at least one cannabinoid comprises a human metabolite of CBD. In some embodiments, the at least one cannabinoid comprises a human metabolite of CBD (see, e.g., Ujvary, I. & Hanus L., Cannabis Cannabinoid Res. 2016; 1 (1): 90-101. the contents of which are incorporated herein by reference in its entirety as relates to human metabolites of CBD). CBD can undergo hydroxylation by CYP450 mixed function oxidases at multiple sites, primarily the liver and gut. Examples of recombinant human CYP enzymes capable of metabolizing CBD include, but are not limited to: CYP1A1, CYP1A2, CYP2C9, CYP2CI9, CYP2D6, CYP3A4, and CYP3A5. As one example, CYP2C19 can metabolize CBD to form the active metabolite 7-hydroxy-cannabidiol (7-OHCBD), which can then be further metabolized by CYP3A4 to an inactive metabolite 7-carboxy-cannabidiol (7-COOH-CBD). The enzymatic processes responsible for the formation of CBD metabolites can also involve several UDP-glucuronosyltransferase (UGT) isoforms, including UGT1A9, UGT2B7 and UGT2B17 and sulfotransferases. In some circumstances, differences in the expression and function of CYP450 enzymes may affect the pharmacokinetics of CBD and its metabolites, which could be relevant in the therapeutic action and any possible adverse effects of CBD-containing preparations.

CBD has been found to be safe for use with both healthy volunteers and in subjects with various medical conditions at doses ranging from 10 mg to 6000 mg administered as both single and multiple doses.

In some embodiments, the at least one cannabinoid (e.g., CBD) is present in the formulation in an amount between 0.1 and 20 wt %, 0.1 and 15 wt %, 0.1 and 10 wt %, 0.1 and 10 wt %, 0.1 and 5 wt %, 0.1 and 1 wt %, 1 and 20 wt %, 1 and 15 wt %, 1 and 10 wt %, 1 and 10 wt %, 1 and 5 wt %, 5 and 20 wt %, 5 and 15 wt %, 5 and 10 wt %, 10 and 20 wt %, 10 and 15 wt %, or 15 and 20 wt %. For example, the at least one cannabinoid may be present in the formulation in an amount of 20, 19.5, 18.5, 18, 17.5, 17, 16.5, 16, 15.5, 15, 14.5, 14, 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, 0.5 or 0.1 wt %.

In some embodiments, the at least one cannabinoid (e.g., CBD) is present in the pharmaceutical composition in an amount between 0.1 and 20 wt %. In some embodiments, the at least one cannabinoid is present in the pharmaceutical composition in an amount between 0.1 and 12 wt %. In some embodiments, the at least one cannabinoid is present in the pharmaceutical composition in an amount between 5 and 12 wt %. In some embodiments, the at least one cannabinoid is present in the pharmaceutical composition in an amount between 4 and 11 wt %. In some embodiments, the at least one cannabinoid is present in the pharmaceutical composition in an amount between 5 and 10 wt %.

In some embodiments, the pharmaceutical composition may comprise at least 1 mg of at least one cannabinoid (e.g., CBD) per capsule or tablet. In some embodiments, the pharmaceutical composition may comprise at least 5 mg, at least 10 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 125 mg, or at least 150 mg of at least one cannabinoid (e.g., CBD) per capsule or tablet.

In some embodiments, the pharmaceutical composition may comprise between 10 and 200 mg of at least one cannabinoid (e.g., CBD) per capsule or tablet. In some embodiments, the pharmaceutical composition may comprise between 10 and 300 mg, 10 and 200 mg, 10 and 195 mg, 10 and 190 mg, 10 and 185 mg, 10 and 180 mg, 10 and 175 mg, 10 and 170 mg, 10 and 165 mg, 10 and 160 mg, 10 and 155 mg, 10 and 150 mg, 10 and 145 mg, 10 and 140 mg, 10 and 135 mg, 10 and 130 mg, 10 and 125 mg, 10 and 120 mg, 10 and 115 mg, 10 and 110 mg, 10 and 105 mg, 10 and 100 mg, 10 and 95 mg, 10 and 90 mg, 10 and 85 mg, 10 and 80 mg, 10 and 75 mg, 10 and 70 mg, 10 and 65 mg, 10 and 60 mg, 10 and 55 mg, 10 and 50 mg, 10 and 45 mg, 10 and 40 mg, 10 and 35 mg, 10 and 30 mg, 10 and 25 mg, 10 and 20 mg, 10 and 15 mg, 15 and 150 mg, 15 and 145 mg, 15 and 140 mg, 15 and 135 mg, 15 and 130 mg, 15 and 125 mg, 15 and 120 mg, 15 and 115 mg, 15 and 110 mg, 15 and 105 mg, 15 and 100 mg, 15 and 95 mg, 15 and 90 mg, 15 and 85 mg, 15 and 80 mg, 15 and 75 mg, 15 and 70 mg, 15 and 65 mg, 15 and 60 mg, 15 and 55 mg, 15 and 50 mg, 15 and 45 mg, 15 and 40 mg, 15 and 35 mg, 15 and 30 mg, 15 and 25 mg, 15 and 20 mg, 20 and 150 mg, 20 and 145 mg, 20 and 140 mg, 20 and 135 mg, 20 and 130 mg, 20 and 125 mg, 20 and 120 mg, 20 and 115 mg, 20 and 110 mg, 20 and 105 mg, 20 and 100 mg, 20 and 95 mg, 20 and 90 mg, 20 and 85 mg, 20 and 80 mg, 20 and 75 mg, 20 and 70 mg, 20 and 65 mg, 20 and 60 mg, 20 and 55 mg, 20 and 50 mg, 20 and 45 mg, 20 and 40 mg, 20 and 35 mg, 20 and 30 mg, 20 and 25 mg, 25 and 150 mg, 25 and 145 mg, 25 and 140 mg, 25 and 135 mg, 25 and 130 mg, 25 and 125 mg, 25 and 120 mg, 25 and 115 mg, 25 and 110 mg, 25 and 105 mg, 25 and 100 mg, 25 and 95 mg, 25 and 90 mg, 25 and 85 mg, 25 and 80 mg, 25 and 75 mg, 25 and 70 mg, 25 and 65 mg, 25 and 60 mg, 25 and 55 mg, 25 and 50 mg, 25 and 45 mg, 25 and 40 mg, 25 and 35 mg, 25 and 30 mg, 30 and 150 mg, 30 and 145 mg, 30 and 140 mg, 30 and 135 mg, 30 and 130 mg, 30 and 125 mg, 30 and 120 mg, 30 and 115 mg, 30 and 110 mg, 30 and 105 mg, 30 and 100 mg, 30 and 95 mg, 30 and 90 mg, 30 and 85 mg, 30 and 80 mg, 30 and 75 mg, 30 and 70 mg, 30 and 65 mg, 30 and 60 mg, 30 and 55 mg, 30 and 50 mg, 30 and 45 mg, 30 and 40 mg, 30 and 35 mg, 35 and 150 mg, 35 and 145 mg, 35 and 140 mg, 35 and 135 mg, 35 and 130 mg, 35 and 125 mg, 35 and 120 mg, 35 and 115 mg, 35 and 110 mg, 35 and 105 mg, 35 and 100 mg, 35 and 95 mg, 35 and 90 mg, 35 and 85 mg, 35 and 80 mg, 35 and 75 mg, 35 and 70 mg, 35 and 65 mg, 35 and 60 mg, 35 and 55 mg, 35 and 50 mg, 35 and 45 mg, 35 and 40 mg, 35 and 150 mg, 35 and 145 mg, 35 and 140 mg, 35 and 135 mg, 35 and 130 mg, 35 and 125 mg, 35 and 120 mg, 35 and 115 mg, 35 and 110 mg, 35 and 105 mg, 35 and 100 mg, 35 and 95 mg, 35 and 90 mg, 35 and 85 mg, 35 and 80 mg, 35 and 75 mg, 35 and 70 mg, 35 and 65 mg, 35 and 60 mg, 35 and 55 mg, 35 and 50 mg, 35 and 45 mg, 35 and 40 mg, 40 and 150 mg, 40 and 145 mg, 40 and 140 mg, 40 and 135 mg, 40 and 130 mg, 40 and 125 mg, 40 and 120 mg, 40 and 115 mg, 40 and 110 mg, 40 and 105 mg, 40 and 100 mg, 40 and 95 mg, 40 and 90 mg, 40 and 85 mg, 40 and 80 mg, 40 and 75 mg, 40 and 70 mg, 40 and 65 mg, 40 and 60 mg, 40 and 55 mg, 40 and 50 mg, 40 and 45 mg, 45 and 150 mg, 45 and 145 mg, 45 and 140 mg, 45 and 135 mg, 45 and 130 mg, 45 and 125 mg, 45 and 120 mg, 45 and 115 mg, 45 and 110 mg, 45 and 105 mg, 45 and 100 mg, 45 and 95 mg, 45 and 90 mg, 45 and 85 mg, 45 and 80 mg, 45 and 75 mg, 45 and 70 mg, 45 and 65 mg, 45 and 60 mg, 45 and 55 mg, 45 and 50 mg, 50 and 150 mg, 50 and 145 mg, 50 and 140 mg, 50 and 135 mg, 50 and 130 mg, 50 and 125 mg, 50 and 120 mg, 50 and 115 mg, 50 and 110 mg, 50 and 105 mg, 50 and 100 mg, 50 and 95 mg, 50 and 90 mg, 50 and 85 mg, 50 and 80 mg, 50 and 75 mg, 50 and 70 mg, 50 and 65 mg, 50 and 60 mg, 50 and 55 mg, 55 and 150 mg, 55 and 145 mg, 55 and 140 mg, 55 and 135 mg, 55 and 130 mg, 55 and 125 mg, 55 and 120 mg, 55 and 115 mg, 55 and 110 mg, 55 and 105 mg, 55 and 100 mg, 55 and 95 mg, 55 and 90 mg, 55 and 85 mg, 55 and 80 mg, 55 and 75 mg, 55 and 70 mg, 55 and 65 mg, 55 and 60 mg, 60 and 150 mg, 60 and 145 mg, 60 and 140 mg, 60 and 135 mg, 60 and 130 mg, 60 and 125 mg, 60 and 120 mg, 60 and 115 mg, 60 and 110 mg, 60 and 105 mg, 60 and 100 mg, 60 and 95 mg, 60 and 90 mg, 60 and 85 mg, 60 and 80 mg, 60 and 75 mg, 60 and 70 mg, 60 and 65 mg, 65 and 150 mg, 65 and 145 mg, 65 and 140 mg, 65 and 135 mg, 65 and 130 mg, 65 and 125 mg, 65 and 120 mg, 65 and 115 mg, 65 and 110 mg, 65 and 105 mg, 65 and 100 mg, 65 and 95 mg, 65 and 90 mg, 65 and 85 mg, 65 and 80 mg, 65 and 75 mg, 65 and 70 mg, 70 and 150 mg, 70 and 145 mg, 70 and 140 mg, 70 and 135 mg, 70 and 130 mg, 70 and 125 mg, 70 and 120 mg, 70 and 115 mg, 70 and 110 mg, 70 and 105 mg, 70 and 100 mg, 70 and 95 mg, 70 and 90 mg, 70 and 85 mg, 70 and 80 mg, 70 and 75 mg, 75 and 150 mg, 75 and 145 mg, 75 and 140 mg, 75 and 135 mg, 75 and 130 mg, 75 and 125 mg, 75 and 120 mg, 75 and 115 mg, 75 and 110 mg, 75 and 105 mg, 75 and 100 mg, 75 and 95 mg, 75 and 90 mg, 75 and 85 mg, 75 and 80 mg, 80 and 150 mg, 80 and 145 mg, 80 and 140 mg, 80 and 135 mg, 80 and 130 mg, 80 and 125 mg, 80 and 120 mg, 80 and 115 mg, 80 and 110 mg, 80 and 105 mg, 80 and 100 mg, 80 and 95 mg, 80 and 90 mg, 80 and 85 mg, 85 and 150 mg, 85 and 145 mg, 85 and 140 mg, 85 and 135 mg, 85 and 130 mg, 85 and 125 mg, 85 and 120 mg, 85 and 115 mg, 85 and 110 mg, 85 and 105 mg, 85 and 100 mg, 85 and 95 mg, 85 and 90 mg, 90 and 150 mg, 90 and 145 mg, 90 and 140 mg, 90 and 135 mg, 90 and 130 mg, 90 and 125 mg, 90 and 120 mg, 90 and 115 mg, 90 and 110 mg, 90 and 105 mg, 90 and 100 mg, 90 and 95 mg, 95 and 150 mg, 95 and 145 mg, 95 and 140 mg, 95 and 135 mg, 95 and 130 mg, 95 and 125 mg, 95 and 120 mg, 95 and 115 mg, 95 and 110 mg, 95 and 105 mg, 95 and 100 mg, 100 and 150 mg, 100 and 145 mg, 100 and 140 mg, 100 and 135 mg, 100 and 130 mg, 100 and 125 mg, 100 and 120 mg, 100 and 115 mg, 100 and 110 mg, 100 and 105 mg, 105 and 150 mg, 105 and 145 mg, 105 and 140 mg, 105 and 135 mg, 105 and 130 mg, 105 and 125 mg, 105 and 120 mg, 105 and 115 mg, 105 and 110 mg, 110 and 150 mg, 110 and 145 mg, 110 and 140 mg, 110 and 135 mg, 110 and 130 mg, 110 and 125 mg, 110 and 120 mg, 110 and 115 mg, 115 and 150 mg, 115 and 145 mg, 115 and 140 mg, 115 and 135 mg, 115 and 130 mg, 115 and 125 mg, 115 and 120 mg, 120 and 150 mg, 120 and 145 mg, 120 and 140 mg, 120 and 135 mg, 120 and 130 mg, 120 and 125 mg, 125 and 150 mg, 125 and 145 mg, 125 and 140 mg, 125 and 135 mg, 125 and 130 mg, 130 and 150 mg, 130 and 145 mg, 130 and 140 mg, 130 and 135 mg, 130 and 130 mg, 130 and 125 mg, 130 and 150 mg, 130 and 145 mg, 130 and 140 mg, 130 and 135 mg, 135 and 150 mg, 135 and 145 mg, 135 and 140 mg, 135 and 135 mg, 135 and 150 mg, 135 and 145 mg, 135 and 140 mg, 140 and 150 mg, 140 and 145 mg, or 145 and 150 mg of at least one cannabinoid (e.g., CBD) per capsule or tablet.

In some embodiments, the pharmaceutical composition may comprise 5 mg of at least one cannabinoid (e.g., CBD) per capsule or tablet. In some embodiments, the pharmaceutical composition may comprise 10 mg of at least one cannabinoid per capsule or tablet. In some embodiments, the pharmaceutical composition may comprise 50 mg of at least one cannabinoid per capsule or tablet. In some embodiments, the pharmaceutical composition may comprise 100 mg of at least one cannabinoid per capsule or tablet.

Methods of measuring bioavailability (e.g., the proportion of an active ingredient which reaches the blood stream of a subject able to perform the intended effect) of a therapeutic compound are well known in the art. In short, such methods may comprise the steps of administering a known amount of an active ingredient to a subject, making blood draws at regular intervals from said subject, measuring the concentration of the active ingredient in said subjects' plasma, and graphing said concentration over time. The process of measuring bioavailability may further comprise determining the area under the plasma concentration versus time curve (AUC) for either a specific period (AUC₀₋ₜ) or extrapolated to infinity (AU_{C0-inf}) and/or determining the maximum plasma concentration of the active ingredient (Cₘₐₓ). Percent (%) bioavailability is determined by comparing the AUC for an active ingredient administered via a non-intravenous route to the intravenously delivered AUC, with the intravenous route assumed to offer 100% bioavailability. The overall bioavailability is considered to increase if the AUC or Cₘₐₓ increases between 2 formulations at the same dose. Additionally, the time at which Cₘₐₓ occurs (Tₘₐₓ) and/or the elimination half-life (T_{1/2}) may also be determined with such a procedure, and formulations which alter these pharmacokinetic properties may be advantageous for the treatment of a given indication.

Studies performed in rats determined the bioavailability of CBD delivered orally to be low (2.8% after a single 10 mg/kg) with a T_{1/2} of 4-5 hrs after a 120 mg/kg dose.

In some embodiments, the AUC or Cₘₐₓ of at least one cannabinoid (i.e., CBD) administered in at least one of the disclosed formulations is increased by at least 3%, at least 5%, at least 7%, at least 10%, at least 15%, at least 20%, at least 25%, or more relative to the cannabinoid administered alone.

In some embodiments the AUC of at least one cannabinoid (i.e., CBD) administered in at least one of the disclosed formulations is at least 35 (ng*h/ml) 2, at least 37 (ng*h/ml) 2, at least 39 (ng*h/ml) 2, at least 41 (ng*h/ml) 2, at least 45 (ng*h/ml) 2, at least 50 (ng*h/ml) 2, at least 100 (ng*h/ml) 2, or more.

In some embodiments the Cₘₐₓ of at least one cannabinoid (i.e., CBD) administered in at least one of the disclosed formulations is at least, 14 (ng/ml) 2, at least 17 (ng/ml) 2, at least 20 (ng/ml) 2, at least 25 (ng/ml) 2, at least 30 (ng/ml) 2, at least 35 (ng/ml) 2, at least 40 (ng/ml) 2, at least 45 (ng/ml) 2, at least 50 (ng/ml) 2, at least 75 (ng/ml) 2, at least 100 (ng/ml) 2, or more.

### Formulations

The present disclosure includes water-soluble lipidic formulations capable of solubilizing a cannabinoid, which retain their water-soluble nature once loaded with the cannabinoid. These formulations, when incorporated into suitable pharmaceutical compositions increase the bioavailability of the cannabinoid over the cannabinoid administered alone and cannabinoids dissolved in pure oils. For example, one formulation herein was shown to increase absorption of CBD by 40% relative to CBD administered as a pure oil solution in a study in healthy volunteers.

Provided herein are formulations capable of solubilizing or otherwise carrying lipophilic active ingredients, thereby increasing their bioavailability relative to the active ingredient (e.g., a cannabinoid) administered alone. In some embodiments, the formulations remain water soluble when loaded with the active ingredient and are suitable for inclusion in pharmaceutical products.

In some embodiments, formulations of the present disclose are water free. In some embodiments, the formulation comprises 0 wt % of water, less than 0.000001 wt % of water, less than 0.00001 wt % of water, less than 0.0001 wt % of water, less than 0.001 wt % of water, less than 0.01 wt % of water, less than 0.1 wt % of water, or less than 1 wt % of water. In some embodiments, the water-free formulations are concentrated formulations or concentrates. In some embodiments, concentrated, water free formulations may later be diluted, in water or other liquids, as needed for effective administration or use according to the present disclosure, or the amount of water in the formulation may increase beyond 1 wt % over time due to hydration by atmospheric water.

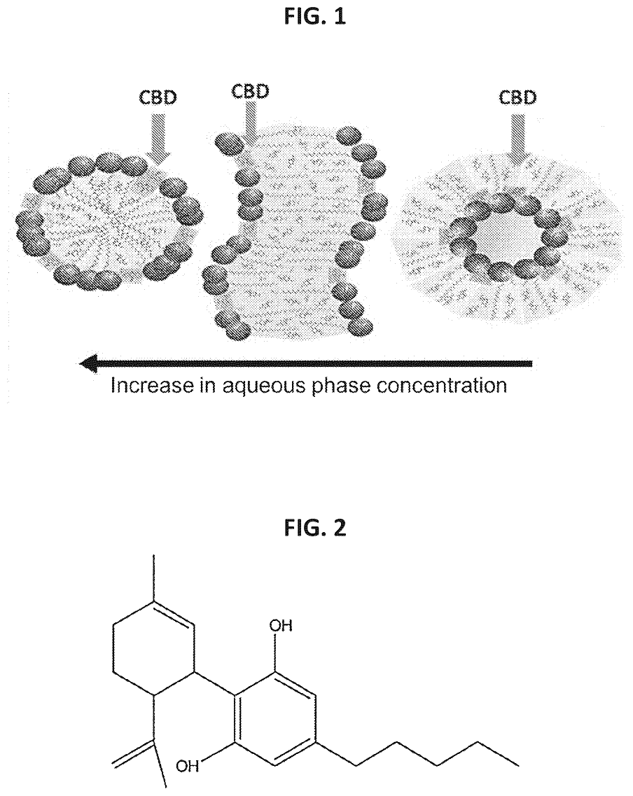

Without being bound by theory, certain combinations of excipients are capable of spontaneous self-assembly into liquid nanodomains when combined in concentrated, water free formulations. These nanodomains, in turn, can serve as carriers for water-insoluble active ingredients (e.g., cannabinoids), rendering them water-soluble. Further, because these nanodomains are very small (10 to 50 nm), they possess a very high surface-area-to-volume ratio, which results in a high loading capacity for the active ingredient. The liquid nanodomains are thermodynamically stable and almost monodispersed. FIG. 1 provides a schematic diagram of said liquid nanodomains loaded with CBD.

Liquid nanodomains also appear to increase the rate of absorption in the gastrointestinal track when administered orally, leading to increased bioavailability of the active ingredient. Without wishing to be bound by theory, the non-ionic surfactants in the formulations of the present disclosure may render CBD less susceptible to degradation or decomposition by the gastric fluid. Phospholipids, when present, likely enhance the mucosal enterocyte's membrane recognition of the nanodomains while medium chain triglyceride or sesame oil components may enhance adherence to the mucosal enterocyte's membrane. The small size of the nanodomains allows for them to spread over a large surface area of the gut and promotes penetration of the mucus-rich “unstirred water layer.” These factors thus provide an increase in bioavailability of the active ingredient due to increased absorption, and a decrease in the time of maximum permeation of the drug.

In some embodiments, liquid nanodomains suitable for use in formulations of the present disclosure can be formed according to the teachings in US 2019/0314326, the content of which is incorporated herein by reference in its entirety, as related to the composition, production, and use of liquid nanodomains suitable for use in formulations of the present disclosure.

In some embodiments, formulations of the present disclosure can comprise at least one cannabinoid, at least one oil, at least one hydrophilic surfactant, and at least one co-surfactant.

In some embodiments, the formulation can optionally comprise at least one solvent, at least one co-solvent, at least one phospholipid, and/or at least one additive.

In some embodiments, formulations of the present disclosure can comprise at least one cannabinoid, at least one oil, at least one surfactant, and at least one co-surfactant, and optionally, at least one solvent, at least one co-solvent, and/or at least one phospholipid.

In some embodiments, formulations of the present disclosure can comprise at least one cannabinoid, at least one oil, at least one hydrophilic surfactant, at least one co-surfactant, and at least one solvent.

In some embodiments, formulations of the present disclosure can comprise at least one cannabinoid, at least one oil, at least one hydrophilic surfactant, at least one co-surfactant, and at least one phospholipid.

In some embodiments, formulations of the present disclosure can comprise at least one cannabinoid, at least one oil, at least one hydrophilic surfactant, at least one co-surfactant, and at least one additive.

In some embodiments, formulations of the present disclosure can comprise at least one cannabinoid, at least one oil, at least one hydrophilic surfactant, at least one co-surfactant, at least one solvent, and at least one co-solvent.

In some embodiments, formulations of the present disclosure can comprise at least one cannabinoid, at least one oil, at least one hydrophilic surfactant, at least one co-surfactant, at least one solvent, and at least one phospholipid.

In some embodiments, formulations of the present disclosure can comprise at least one cannabinoid, at least one oil, at least one hydrophilic surfactant, at least one co-surfactant, at least one solvent, and at least one additive.

In some embodiments, formulations of the present disclosure can comprise at least one cannabinoid, at least one oil, at least one hydrophilic surfactant, at least one co-surfactant, at least one phospholipid, and at least one additive.

In some embodiments, formulations of the present disclosure can comprise at least one cannabinoid, at least one oil, at least one hydrophilic surfactant, at least one co-surfactant, at least one solvent, at least one co-solvent and at least one phospholipid.

In some embodiments, formulations of the present disclosure can comprise at least one cannabinoid, at least one oil, at least one hydrophilic surfactant, at least one co-surfactant, at least one solvent, at least one co-solvent and at least one additive.

In some embodiments, formulations of the present disclosure can comprise at least one cannabinoid, at least one oil, at least one hydrophilic surfactant, at least one co-surfactant, at least one solvent, at least one co-solvent, at least one phospholipid, and at least one additive.

In some embodiments, formulations of the present disclosure can be formed by: (i) combining an oil, a surfactant, a co-surfactant, and optionally, a solvent, a co-solvent, and/or a phospholipid; and (ii) mixing the formulation components until a homogenous, clear (i.e., transparent) mixture is obtained. In cases where the surfactants and/or oil are solid at room temperature, heating can be applied while mixing to allow full dissolution and formation of the formulation. In some embodiments, pharmaceutical compositions of the present disclosure can be formed by combining (e.g., slowly adding) the formulation mixture to a cannabinoid source, followed by appropriate wetting, mixing, and/or homogenization.

In some embodiments, the formulation comprises one or more oils. in some embodiments the one or more oils may be either a synthetic or natural oil. In some embodiments, the oil may include, but is not limited to, medium-chain triglycerides (MCT), sesame oil, seed oils, nut oils, vegetable oils, olive oil, soybean oil, canola oil, cotton oil, palmolein, sunflower oil, corn oil, rapeseed oil, grape seeds oil, hemp oil, pomegranate oil, avocado oil, peppermint oil, tomato oil, isopropyl myristate, oleyl lactate, coco caprylocaprate, hexyl laurate, oleyl amine, oleic acid, oleyl alcohol, linoleic acid, linoleyl alcohol, ethyl oleate, hexane, heptanes, nonane, decane, dodecane, D-limonene, neem oil, lavender oil, peppermint oil, anise oil, rosemary oil, sage oil, hibiscus oil, berries oil (any type), menthol, capsaicin, grape seed oil, pumpkin oil, hemp oil, similar essential oils, triglycerides, esters of fatty acids, and mixtures thereof. In some embodiments, the formulation comprises at least one oil which comprises medium-chain triglycerides (MCT). In some embodiments, the formulation comprises at least one oil which comprises sesame oil. In some embodiments, the formulation comprises at least one oil which comprises medium-chain triglycerides (MCT) and sesame oil.

In some embodiments, the one or more oils may be present in the formulation at an amount of between 0.5 and 20 wt %, 0.5 and 18 wt %, 0.5 and 16 wt %, 0.5 and 14 wt %, 0.5 and 12 wt %, 0.5 and 10 wt %, 0.5 and 8 wt %, 1 and 20 wt %, 1 and 18 wt %, 1 and 16 wt %, 1 and 14 wt %, 1 and 12 wt %, 1 and 10 wt %, 1 and 8 wt %, 2 and 20 wt %, 2 and 18 wt %, 2 and 16 wt %, 2 and 14 wt %, 2 and 12 wt %, 2 and 10 wt %, 2 and 8 wt %, 4 and 20 wt %, 4 and 18 wt %, 4 and 16 wt %, 4 and 14 wt %, 4 and 12 wt %, 4 and 10 wt %, 4 and 8 wt %, 6 and 20 wt %, 6 and 18 wt %, 6 and 16 wt %, 6 and 14 wt %, 6 and 12 wt %, 6 and 10 wt %, 6 and 8 wt %, 8 and 20 wt %, 8 and 18 wt %, 8 and 16 wt %, 8 and 14 wt %, 8 and 12 wt %, 8 and 10 wt %, 10 and 20 wt %, 10 and 18 wt %, 10 and 16 wt %, 10 and 14 wt %, 10 and 12 wt %, 12 and 20 wt %, 12 and 18 wt %, 12 and 16 wt %, 12 and 14 wt %, 14 and 20 wt %, 14 and 18 wt %, 14 and 16 wt %, 16 and 20 wt %, 16 and 18 wt %, or 18 and 20 wt %.

In some embodiments, the one or more oils may be present in the formulation at an amount between 0.5 and 20 wt %. In other embodiments, the one or more oils may be present in the formulation at an amount between 1 and 10 wt %. In other embodiments the one or more oils may be present in the formulation in an amount between 3 and 6 wt %.

In some embodiments, the one or more oils may be present in the formulation in a wt % of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 15, 16.5, 17, 17.5, 18, 18.5, 19, 19.5, or 20 wt %.

In some embodiments the one or more oils may be present in an amount of 3 wt %. In some embodiments the one or more oils may be present in an amount of 4 wt %. In some embodiments the at least one oil may be present in an amount of 5 wt %. In some embodiments the one or more oils may be present in an amount of 6 wt %. In some embodiments the one or more oils may be present in an amount of 11 wt %.

In some embodiments, the amount oil present in the formulation may be measured as the mass of the one or more oils present in one (1) tablet or capsule of the pharmaceutical composition. In such embodiments, the amount of the one or more oils may be between 5 and 200 mg per one (1) tablet or capsule. In some embodiments, the one or more oils may be present in an amount between 5 and 10 mg, 5 and 20 mg, 5 and 30 mg, 5 and 40 mg, 5 and 50 mg, 5 and 60 mg, 5 and 70 mg, 5 and 80 mg, 5 and 90 mg, 5 and 100 mg, 5 and 110 mg, 5 and 120 mg, 5 and 130 mg, 5 and 140 mg, 5 and 150 mg, 5 and 160 mg, 5 and 170 mg, 5 and 180 mg, 5 and 190 mg, 10 and 20 mg, 10 and 30 mg, 10 and 40 mg, 10 and 50 mg, 10 and 60 mg, 10 and 70 mg, 10 and 80 mg, 10 and 90 mg, 10 and 100 mg, 10 and 110 mg, 10 and 120 mg, 10 and 130 mg, 10 and 140 mg, 10 and 150 mg, 10 and 160 mg, 10 and 170 mg, 10 and 180 mg, 10 and 190 mg, 10 and 200 mg, 20 and 30 mg, 20 and 40 mg, 20 and 50 mg, 20 and 60 mg, 20 and 70 mg, 20 and 80 mg, 20 and 90 mg, 20 and 100 mg, 20 and 110 mg, 20 and 120 mg, 20 and 130 mg, 20 and 140 mg, 20 and 150 mg, 20 and 160 mg, 20 and 170 mg, 20 and 180 mg, 20 and 190 mg, 20 and 200 mg, 30 and 40 mg, 30 and 50 mg, 30 and 60 mg, 30 and 70 mg, 30 and 80 mg, 30 and 90 mg, 30 and 100 mg, 30 and 110 mg, 30 and 120 mg, 30 and 130 mg, 30 and 140 mg, 30 and 150 mg, 30 and 160 mg, 30 and 170 mg, 30 and 180 mg, 30 and 190 mg, 30 and 200 mg, 40 and 50 mg, 40 and 60 mg, 40 and 70 mg, 40 and 80 mg, 40 and 90 mg, 40 and 100 mg, 40 and 110 mg, 40 and 120 mg, 40 and 130 mg, 40 and 140 mg, 40 and 150 mg, 40 and 160 mg, 40 and 170 mg, 40 and 180 mg, 40 and 190 mg, 40 and 200 mg, 50 and 60 mg, 50 and 70 mg, 50 and 80 mg, 50 and 90 mg, 50 and 100 mg, 50 and 110 mg, 50 and 120 mg, 50 and 130 mg, 50 and 140 mg, 50 and 150 mg, 50 and 160 mg, 50 and 170 mg, 50 and 180 mg, 50 and 190 mg, 50 and 200 mg, 60 and 70 mg, 60 and 80 mg, 60 and 90 mg, 60 and 100 mg, 60 and 110 mg, 60 and 120 mg, 60 and 130 mg, 60 and 140 mg, 60 and 150 mg, 60 and 160 mg, 60 and 170 mg, 60 and 180 mg, 60 and 190 mg, 60 and 200 mg, 70 and 80 mg, 70 and 90 mg, 70 and 100 mg, 70 and 110 mg, 70 and 120 mg, 70 and 130 mg, 70 and 140 mg, 70 and 150 mg, 70 and 160 mg, 70 and 170 mg, 70 and 180 mg, 70 and 190 mg, 70 and 200 mg, 80 and 90 mg, 80 and 100 mg, 80 and 110 mg, 80 and 120 mg, 80 and 130 mg, 80 and 140 mg, 80 and 150 mg, 80 and 160 mg, 80 and 170 mg, 80 and 180 mg, 80 and 190 mg, 80 and 200 mg, 90 and 100 mg, 90 and 110 mg, 90 and 120 mg, 90 and 130 mg, 90 and 140 mg, 90 and 150 mg, 90 and 160 mg, 90 and 170 mg, 90 and 180 mg, 90 and 190 mg, 90 and 200 mg, 100 and 110 mg, 100 and 120 mg, 100 and 130 mg, 100 and 140 mg, 100 and 150 mg, 100 and 160 mg, 100 and 170 mg, 100 and 180 mg, 100 and 190 mg, 100 and 200 mg, 110 and 120 mg, 110 and 130 mg, 110 and 140 mg, 110 and 150 mg, 110 and 160 mg, 110 and 170 mg, 110 and 180 mg, 110 and 190 mg, 110 and 200 mg, 120 and 130 mg, 120 and 140 mg, 120 and 150 mg, 120 and 160 mg, 120 and 170 mg, 120 and 180 mg, 120 and 190 mg, 120 and 200 mg, 130 and 140 mg, 130 and 150 mg, 130 and 160 mg, 130 and 170 mg, 130 and 180 mg, 130 and 190 mg, 130 and 200 mg, 140 and 150 mg, 140 and 160 mg, 140 and 170 mg, 140 and 180 mg, 140 and 190 mg, 140 and 200 mg, 150 and 160 mg, 150 and 170 mg, 150 and 180 mg, 150 and 190 mg, 150 and 200 mg, 160 and 170 mg, 160 and 180 mg, 160 and 190 mg, 160 and 200 mg, 170 and 180 mg, 170 and 190 mg, 170 and 200 mg, 180 and 190 mg, 180 and 200 mg, or between 190 and 200 mg per one (1) tablet or capsule.

In some embodiments, the amount of the one or more oils may be between 50 and 60 mg per one (1) tablet or capsule. In some embodiments the one or more oils are present in an amount between 50 and 60 mg, 51 and 60 mg, 52 and 60 mg, 53 and 60 mg, 54 and 60 mg, 55 and 60 mg, 56 and 60 mg, 57 and 60 mg, 58 and 60 mg, 59 and 60 mg, 50 and 59 mg, 51 and 59 mg, 52 and 59 mg, 53 and 59 mg, 54 and 59 mg, 55 and 59 mg, 56 and 59 mg, 57 and 59 mg, 58 and 59 mg, 50 and 58 mg, 51 and 58 mg, 52 and 58 mg, 53 and 58 mg, 54 and 58 mg, 55 and 58 mg, 56 and 58 mg, 57 and 58 mg, 50 and 57 mg, 51 and 57 mg, 52 and 57 mg, 53 and 57 mg, 54 and 57 mg, 55 and 57 mg, 55 and 57 mg, 56 and 57 mg, 50 and 56 mg, 51 and 56 mg, 52 and 56 mg, 53 and 56 mg, 54 and 56 mg, 55 and 56 mg, 50 and 55 mg, 51 and 55 mg, 52 and 55 mg, 53 and 55 mg, 54 and 55 mg, 50 and 54 mg, 51 and 54 mg, 52 and 54 mg, 53 and 54 mg, 50 and 53 mg, 51 and 53 mg, 52 and 53 mg, 50 and 52 mg, 51 and 52 mg, or between 50 and 51 mg per one (1) tablet or capsule.

In some embodiments, the amount of the one or more oils may be 54 mg per one (1) tablet or capsule. In some embodiments, the amount of the one or more oils may be 57 mg per one (1) tablet or capsule. In some embodiments, the amount of the one or more oils may be 60 mg per one (1) tablet or capsule. In some embodiments, the amount of the one or more oils may be 110 mg per one (1) tablet or capsule.

In some embodiments, the formulation comprises at least one hydrophilic surfactant. In some embodiments, the hydrophilic surfactant may include, but is not limited to, polyoxyethylenes, ethoxylated (20EO) sorbitan mono laurate (T20), ethoxylated (20EO) sorbitan monostearate/palmitate (T60), ethoxylated (20EO) sorbitan mono oleate/linoleate (TSO), ethoxylated (20EO) sorbitan trioleate (T85), castor oil ethoxylated (20EO to 40EO); hydrogenated castor oil ethoxylated (20 to 40EO), ethoxylated (5-40 EO) monoglyceride stearate/palmitate, PEG-8 caprylic/capric glycerides (oleoyl macrogolglycerides, e.g., Labrasol® ALF), polyoxyl 35 castor oil (e.g., Cremophor EL), Solutol HS15 (Polyethylene glycol (15)-hydroxystearatepolysorbate 40 (e.g., Tween 40), polysorbate 60 (e.g., Tween 60), polysorbate 80 (e.g., Tween 80), Mirj S40, oleoyl macrogolglycerides, polyglyceryl-3 dioleate, ethoxylated, hydroxystearate, polyglycerol esters such as decaglycerol monolaurate, decaglycerol. monooleate, hexaglycerol monooleate and hexaglycerol monolaurate, sucrose monooleate, sucrose monolaurate, ethoxylated monglycerol esters, ethoxylated fatty acids and ethoxylated fatty acids of short and medium and long chain fatty acids. In some embodiments, the hydrophilic surfactant comprises polyoxyl 35 castor oil (e.g., Cremophor EL). In some embodiments, the hydrophilic surfactant comprises Polysorbate 80. In some embodiments, the hydrophilic surfactant comprises PEG-8 caprylic/capric glycerides.

In some embodiments, the formulation may comprise between 30 and 85 wt %, 30 and 35 wt %, 30 and 40 wt %, 30 and 45 wt %, 30 and 50 wt %, 30 and 55 wt %, 30 and 60 wt %, 30 and 65 wt %, 30 and 70 wt %, 30 and 75 wt %, 30 and 80 wt %, 30 and 85 wt %, 35 and 40 wt %, 35 and 45 wt %, 35 and 50 wt %, 35 and 55 wt %, 35 and 60 wt %, 35 and 65 wt %, 35 and 70 wt %, 35 and 75 wt %, 35 and 80 wt %, 35 and 85 wt %, 40 and 45 wt %, 40 and 50 wt %, 40 and 55 wt %, 40 and 60 wt %, 40 and 65 wt %, 40 and 70 wt %, 40 and 75 wt %, 40 and 80 wt %, 40 and 85 wt %, 45 and 50 wt %, 45 and 55 wt %, 45 and 60 wt %, 45 and 65 wt %, 45 and 70 wt %, 45 and 75 wt %, 45 and 80 wt %, 45 and 85 wt %, 50 and 55 wt %, 50 and 60 wt %, 50 and 65 wt %, 50 and 70 wt %, 50 and 75 wt %, 50 and 80 wt %, 50 and 85 wt %, 55 and 60 wt %, 55 and 65 wt %, 55 and 70 wt %, 55 and 75 wt %, 55 and 80 wt %, 55 and 85 wt %, 60 and 65 wt %, 60 and 70 wt %, 60 and 75 wt %, 60 and 80 wt %, 60 and 85 wt %, 65 and 70 wt %, 65 and 75 wt %, 65 and 80 wt %, 65 and 85 wt %, 70 and 75 wt %, 70 and 80 wt %, 70 and 85 wt %, 75 and 80 wt %, 75 and 85 wt %, or between 80 and 85 wt %, of said at least one hydrophilic surfactant.

In some embodiments, the formulation may comprise, between 30 and 85 wt % of at least one hydrophilic surfactant. In some other embodiments, the formulation may comprise between 35 and 80 wt % of at least one hydrophilic surfactant. In some embodiments, the formulation may comprise between 45 and 80 wt % of at least one hydrophilic surfactant. In some embodiments, the formulation may comprise between 45 and 55 wt % of at least one hydrophilic surfactant. In some embodiments, the formulation may comprise between 70 and 80 wt % of at least one hydrophilic surfactant.

In some embodiments, the formulation may comprise 45, 46, 47, 48, 49, 50, 51, 52, 53, 54, or 55 wt % of at least one hydrophilic surfactant. In some embodiments, the formulation may comprise 70, 71, 72, 73, 74, 75, 76, 77, 78, 79, or 80 wt % of at least one hydrophilic surfactant. In some embodiments, the formulation may comprise 38 wt % of at least one hydrophilic surfactant. In some embodiments, the formulation may comprise 28 wt % of at least one hydrophilic surfactant. In some embodiments, the formulation may comprise 48 wt % of at least one hydrophilic surfactant. In some embodiments, the formulation may comprise 12 wt % of at least one hydrophilic surfactant.

In some embodiments, the amount of at least one hydrophilic surfactant present in the formulation may be measured as the mass of the at least one hydrophilic surfactant present in one (1) tablet or capsule of the pharmaceutical composition. In such embodiments, the amount of said at least one hydrophilic surfactant may be between 300 and 850 mg per one (1) tablet or capsule. For example, in some embodiments said at least one hydrophilic surfactant may be present in amounts between 300 and 800 mg, 300 and 750 mg, 300 and 700 mg, 300 and 650 mg, 300 and 600 mg, 300 and 550 mg, 300 and 500 mg, 300 and 450 mg, 300 and 400 mg, 300 and 350 mg, 350 and 850 mg, 350 and 800 mg, 350 and 750 mg, 350 and 700 mg, 350 and 650 mg, 350 and 600 mg, 350 and 550 mg, 350 and 500 mg, 350 and 450 mg, 350 and 400 mg, 400 and 850 mg, 400 and 800 mg, 400 and 750 mg, 400 and 700 mg, 400 and 650 mg, 400 and 600 mg, 400 and 550 mg, 400 and 500 mg, 400 and 450 mg, 450 and 850 mg, 450 and 800 mg, 450 and 750 mg, 450 and 700 mg, 450 and 650 mg, 450 and 600 mg, 400 and 550 mg, 400 and 500 mg, 400 and 450 mg, 450 and 850 mg, 450 and 800 mg, 450 and 750 mg, 450 and 700 mg, 450 and 650 mg, 450 and 600 mg, 450 and 550 mg, 450 and 500 mg, 500 and 850 mg, 500 and 800 mg, 500 and 750 mg, 500 and 700 mg, 500 and 650 mg, 500 and 600 mg, 500 and 550 mg, 550 and 850 mg, 550 and 800 mg, 550 and 750 mg, 550 and 700 mg, 550 and 650 mg, 550 and 600 mg, 600 and 850 mg, 600 and 800 mg, 600 and 750 mg, 600 and 700 mg, 600 and 650 mg, 650 and 850 mg, 650 and 750 mg, 650 and 700 mg, 700 and 850 mg, 700 and 800 mg, 700 and 750 mg, 750 and 850 mg, 750 and 800 mg, or 800 and 850 mg per one (1) tablet or capsule. In some embodiments the amount of said at least one hydrophilic surfactant may be between 100 and 300 mg per one (1) tablet or capsule. For example, in some embodiments said at least one hydrophilic surfactant may be present in amounts between 100 and 150 mg, 100 and 200 mg, 100 and 250 mg, 100 and 300 mg, 150 and 200 mg, 150 and 250 mg, 150 and 300 mg, 200 and 250 mg, 200 and 300 mg, or 250 and 300 mg per one (1) tablet or capsule.

In some embodiments, the amount of at least one hydrophilic surfactant present in the composition may be between 700 and 800 mg per one (1) tablet or capsule. For example, in some embodiments said at least one hydrophilic surfactant may be present in an amount of 700, 705, 710, 715, 720, 725, 730, 735, 740, 745, 750, 755, 760, 765, 770, 775, 780, 785, 790, 795, or 800 mg per one (1) tablet or capsule.

In some embodiments, the amount of at least one hydrophilic surfactant present in the composition may be 715 mg per one (1) tablet or capsule. In some embodiments, the amount of at least one hydrophilic surfactant present in the composition may be 755 mg per one (1) tablet or capsule. In some embodiments, the amount of at least one hydrophilic surfactant present in the composition may be 795 mg per one (1) tablet or capsule. In some embodiments, the amount of at least one hydrophilic surfactant present in the composition may be 380 mg per one (1) tablet or capsule. In some embodiments, the amount of at least one hydrophilic surfactant present in the composition may be 280 mg per one (1) tablet or capsule. In some embodiments, the amount of at least one hydrophilic surfactant present in the composition may be 484 mg per one (1) tablet or capsule. In some embodiments, the amount of at least one hydrophilic surfactant present in the composition may be 115 mg per one (1) tablet or capsule. In some embodiments, the amount of at least one hydrophilic surfactant present in the composition may be 116 mg per one (1) tablet or capsule.

In some embodiments, the formulation may comprise at least one co-surfactant. In some embodiments, the co-surfactant may comprise, but is not limited to, at least one polyol, i.e., an alcohol containing at least 2 hydroxyl groups, for example ethylene glycol, glycerol, polyethylene glycol, polypropylene glycol, sorbitol, mannitol, lactitol, xylitol and others. In some embodiments, the co-surfactant may be selected from glycerol, polypropylene glycol, polyethylene glycol, Propylene Glycol, Polyglyceryl-3 oleate (Plurol® Oleique CC 947), ethoxy hydrogenated castor oil, sorbitan esters of saturated or unsaturated fatty acids (Spans), phospholipids, waxes (carnauba, beeswax, candellila).

In some embodiments, the formulation may comprise between 1 and 50 wt %, 1 and 45 wt %, 1 and 40 wt %, 1 and 35 wt %, 1 and 30 wt %, 1 and 25 wt %, 1 and 20 wt %, 1 and 15 wt %, 1 and 10 wt %, 1 and 5 wt %, 5 and 50 wt %, 5 and 45 wt %, 5 and 40 wt %, 5 and 35 wt %, 5 and 30 wt %, 5 and 25 wt %, 5 and 20 wt %, 5 and 15 wt %, 5 and 10 wt %, 5 and 5 wt %, 10 and 50 wt %, 10 and 45 wt %, 10 and 40 wt %, 10 and 35 wt %, 10 and 30 wt %, 10 and 25 wt %, 10 and 20 wt %, 10 and 15 wt %, 15 and 50 wt %, 15 and 45 wt %, 15 and 40 wt %, 15 and 35 wt %, 15 and 30 wt %, 15 and 25 wt %, 15 and 20 wt %, 20 and 50 wt %, 20 and 45 wt %, 20 and 30 wt %, 20 and 25 wt %, 25 and 50 wt %, 25 and 45 wt %, 25 and 40 wt %, 25 and 35 wt %, 25 and 30 wt %, 30 and 50 wt %, 30 and 45 wt %, 30 and 40 wt %, 30 and 35 wt %, 35 and 50 wt %, 35 and 45 wt %, 35 and 40 wt %, 40 and 50 wt %, 40 and 45 wt %, or 45 and 50 wt % of the at least one co-surfactant.

In some embodiments, the formulation may comprise between 1 and 50 wt % of the at least one co-surfactant. In other embodiments, the formulation may comprise between 2 and 45 wt % of the at least one co-surfactant. In still more embodiments, the formulation may comprise between 2 and 5 wt % of the at least one co-surfactant.

In some embodiments, the co-surfactant is present in the formulation at an amount from between 1 and 50 wt %. In other embodiments, the co-surfactant may be present in the formulation in an amount of between 2 and 45 wt %. In other embodiments, the co-surfactant may be present in the formulation in an amount of between 2 and 5 wt %.

In some embodiments, the formulation may comprise 2, 3, 4, 5, 6, 7, 8, 9, 10, 11 12, 13, or 14 wt % of the at least one co-surfactant. In some embodiments, the formulation may comprise 4 wt % of the at least one co-surfactant. In some embodiments, the formulation may comprise 8 wt % of the at least one co-surfactant. In some embodiments, the formulation may comprise 3 wt % of the at least one co-surfactant. In some embodiments, the formulation may comprise 14 wt % of the at least one co-surfactant.

In some embodiments, the amount of at least one co-surfactant present in the formulation may be measured as the mass of the at least one hydrophilic surfactant present in one (1) tablet or capsule of the pharmaceutical composition. In such embodiments, the amount of said at least one co-surfactant may be between 10 and 500 mg per one (1) tablet or capsule. For example, in some embodiments said at least one co-surfactant may be present in amounts between 10 and 500 mg, 10 and 450 mg, 10 and 400 mg, 10 and 350 mg, 10 and 300 mg, 10 and 250 mg, 10 and 200 mg, 10 and 150 mg, 10 and 100 mg, 10 and 50 mg, 50 and 500 mg, 50 and 450 mg, 50 and 400 mg, 50 and 350 mg, 50 and 300 mg, 50 and 250 mg, 50 and 200 mg, 50 and 150 mg, 50 and 100 mg, 100 and 500 mg, 100 and 450 mg, 100 and 400 mg, 100 and 350 mg, 100 and 300 mg, 100 and 250 mg, 100 and 200 mg, 100 and 150 mg, 150 and 500 mg, 150 and 450 mg, 150 and 400 mg, 150 and 350 mg, 150 and 300 mg, 150 and 250 mg, 150 and 200 mg, 200 and 500 mg, 200 and 450 mg, 200 and 400 mg, 200 and 350 mg, 200 and 300 mg, 200 and 250 mg, 250 and 500 mg, 250 and 450 mg, 250 and 400 mg, 250 and 350 mg, 250 and 300 mg, 300 and 500 mg, 300 and 450 mg, 300 and 400 mg, 300 and 350 mg, 350 and 500 mg, 350 and 450 mg, 350 and 400 mg, 400 and 500 mg, 400 and 450 mg, or 450 and 500 mg per one (1) tablet or capsule.

In some embodiments, the at least one co-surfactant may be present in the composition in an amount between 20 and 50 mg per one (1) tablet or capsule. For example, the at least one co-surfactant may be present in the composition in an amount of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per one (1) tablet or capsule. In some embodiments, the at least one co-surfactant may be present in the composition in an amount between 25 and 150 mg per one (1) tablet or capsule.

In some embodiments, the at least one co-surfactant may be present in the composition in an amount of 45 mg per one (1) tablet or capsule. In other embodiments, the at least one co-surfactant may be present in the composition in an amount of 85 mg per one (1) tablet or capsule. In still more embodiments, the at least one co-surfactant may be present in the composition in an amount of 140 mg per one (1) tablet or capsule. In some embodiments, the at least one co-surfactant may be present in the composition in an amount of 30 mg per one (1) tablet or capsule.

In some embodiments, the formulation may contain at least one solvent. In some embodiments the at least one solvent may be but is not limited to an organic compound, different from the oil, which is miscible in the oil and together therewith form a homogenous oily phase that dissolves and stabilizes the cannabinoid.

In some embodiments, the solvent may be selected from, but is not limited to, ethanol, propanol, isopropyl alcohol, acetic acid, propionic acid, fumaric acid, tartaric acid and its derivatives, lactic acid, maleic acid, and malic acid.

In some embodiments, the at least one solvent may be present in the formulation in an amount between 0.1 and 25 wt %, 0.1 and 20 wt %, 0.1 and 15 wt %, 0.1 and 10 wt %, 0.1 and 5 wt %, 1 and 25 wt %, 1 and 20 wt %, 1 and 15 wt %, 1 and 10 wt %, 1 and 5 wt %, 5 and 25 wt %, 5 and 20 wt %, 5 and 15 wt %, 5 and 10 wt %, 10 and 25 wt %, 10 and 20 wt %, 10 and 15 wt %, 15 and 25 wt %, 15 and 20 wt %, or 20 and 25 wt %.

In some embodiments, the at least one solvent may be present in the formulation at in an amount between 0.1 and 25 wt %. In some embodiments, the formulation may comprise between 0.1 and 15 wt % of the at least one solvent.

In some embodiments, the amount of at least one solvent present in the formulation may be measured as the mass of the at least one solvent present in one (1) tablet or capsule of the pharmaceutical composition. In such embodiments, the amount of said at least one solvent may be between 1 and 250 mg per one (1) tablet or capsule. For example, in some embodiments said at least one solvent may be present in amounts between 1 and 250 mg, 1 and 200 mg, 1 and 150 mg, 1 and 100 mg, 1 and 50 mg, 10 and 250 mg, 10 and 200 mg, 10 and 150 mg, 10 and 100 mg, 10 and 50 mg, 50 and 250 mg, 50 and 200 mg, 50 and 150 mg, 50 and 100 mg, 100 and 250 mg, 100 and 200 mg, 100 and 150 mg, 150 and 250 mg, 150 and 200 mg, or 200 and 250 mg per one (1) tablet or capsule.

In some embodiments, the formulation may contain at least one phospholipid. In some embodiments, the phospholipids may be selected from, but are not limited to, soy lecithin, rapeseed lecithin, corn or sunflower lecithins, egg lecithin, Epicom 200, Phosal 50 PG, dioleyl phospatidylcholine (DOPC), oleyl palmytoyl phosphatidylcholine (POPC), and the corresponding serines, ethanol amines, glycerol, and others.

In some embodiments, the least one phospholipid may comprise between 1 and 10 wt % of the formulation. In some embodiments, the least one phospholipid may be present in the formulation in an amount of 1, 2, 3, 4, 5, 6, 7, 8, 9, or 10 wt %.

In some embodiments, the amount of at least one phospholipid present in the formulation may be measured as the mass of the at least one phospholipid present in one (1) tablet or capsule of the pharmaceutical composition. In some embodiments, the amount of the at least one phospholipid may be between 10 and 100 mg per one (1) tablet or capsule. In some embodiments, the at least one phospholipid may be present in amounts between 10 and 100 mg, 10 and 80 mg, 10 and 60 mg, 10 and 40 mg, 10 and 20 mg, 20 and 100 mg, 20 and 80 mg, 20 and 60 mg, 20 and 40 mg, 40 and 100 mg, 40 and 80 mg, 40 and 60 mg, 60 and 100 mg, 60 and 80 mg, or 80 and 100 mg per one (1) tablet or capsule.

In some embodiments, the formulation may comprise at least one additive, selected from antioxidants (e.g., tocopherols), preservatives, membrane-piercing agents, transmembrane penetrating enhancers (such as transcutol, isosorbide, oleic acid, propylene glycol, maltodextrines, cyclodextrines, etc.), oil/water soluble vitamins, BHA, BHT, TBHQ, Propylate and its derivatives, and others.

In some embodiments, the at least one additive may be present in the formulation in an amount of between 0.01 and 15 wt %, 0.01 and 10 wt %, 0.01 and 5 wt %, 1 and 15 wt %, 1 and 10 wt %, 1 and 5 wt %, 5 and 15 wt %, 5 and 10 wt %, or 10 and 15 wt %.

In some embodiments, the at least one additive may be present in the formulation in an amount of between 0.01 and 10 wt %. In some embodiments, the at least one additive may be present in the formulation in an amount of between 5 and 7 wt %. In some other embodiments, the at least one additive may be present in the invention in an amount of between 8 and 10 wt %. In some embodiments, the at least one additive may be present in the formulation in an amount of between 0.01 and 5 wt %. In some embodiments, the at least one additive may be present in the formulation in an amount of between 0.05 wt %.

In some embodiments, the amount of at least one additive present in the formulation may be measured as the mass of the at least one additive present in one (1) tablet or capsule of the pharmaceutical composition. In some embodiments, the amount of the at least one additive may be between 1 and 150 mg per one (1) tablet or capsule. In some embodiments, the least one additive may be present in amounts between 1 and 150 mg, 1 and 100 mg, 1 and 50 mg, 10 and 150 mg, 10 and 100 mg, 10 and 50 mg, 50 and 150 mg, 50 and 100 mg, or 100 and 150 mg per one (1) tablet or capsule.

In some embodiments, the at least one additive may be present in the composition in an amount between 0.1 and 5 mg per one (1) tablet or capsule.

In some embodiments, the at least one additive may be present in the composition in an amount of 0.5 mg per one (1) tablet or capsule.

In some embodiments, the formulation may comprise: (i) at least one cannabinoid; (ii) at least one oil selected from medium chain triglyceride (MCT), sesame oil, glycerin, glycerol, castor oil, R (+)-limonene, isopropyl myristate, ethyl laurate, ethyl caprate, olive oil, oleic acid, and triacetin; (iii) at least one hydrophilic surfactant selected from polysorbate 80 (e.g., Tween 80), polyoxyl 35 castor oil (cremophor castor oil), Mirj S40, HECO40 (ethoxy 40 hydrogenated castor oil), PEG-8 caprylic/capric glycerides (oleoyl macrogolglycerides, e.g., Labrasol® ALF), glycerol, and sucrose mono/dilaurate; (iv) at least one co-surfactant selected from polyglyceryl-3 oleate, polypropylene glycol (PG), Propylene Glycol, and Plural Oleique CC 497 (Polyglyceryl-3 dioleate); (v) at least one additive selected from propylene glycol beta hydroxy acid (BHA), butylated hydroxytoluene (BHT), or tertiary butylhydroquinone (TBHQ); (vi) at least one phospholipid; (vii) at least one solvent selected from oleic acid, transcutol, acetic acid, ethanol and isopropyl alcohol; or any combination thereof.

In some embodiments, the formulation may comprise MCT, sesame oil, polyoxyl 35 castor oil, polysorbate 80, PEG-8 caprylic/capric glycerides, polyglyceryl-3 oleate, propylene glycol, BHT, or any combination thereof.

In some embodiments, the formulation may comprise one or more formulation component as disclosed in US 2019/0314326, the content of which is incorporated herein by reference in its entirety as related to composition, production, and use of formulations suitable for use in present disclosure. In some embodiments, the formulation may comprise one or more formulation mixtures selected from: medium chain triglyceride (MCT), polysorbate 80 (Tween 80), polyoxyl 35 castor oil (cremophor castor oil), polypropylene glycol (PG), ethanol, and at least one phospholipid; or medium chain triglyceride (MCT), glycerin, polysorbate 80 (Tween 80), polyoxyl 35 castor oil (cremophor castor oil), polypropylene glycol (PG), ethanol, and at least one phospholipid; or medium chain triglyceride (MCT), oleic acid, polysorbate 80 (Tween 80), polyoxyl 35 castor oil (cremophor castor oil), polypropylene glycol (PG), ethanol, and at least one phospholipid; or R-(+)-limonene, polysorbate 80 (Tween 80), polypropylene glycol (PG), and ethanol; or R-(+)-limonene, polysorbate 80 (Tween 80), polyoxyl 35 castor oil (cremophor castor oil), and polypropylene glycol (PG); or medium chain triglycerides (MCT), polysorbate 80 (Tween 80), polyoxyl 35 castor oil (cremophor castor oil), and polypropylene glycol (PG); or—isopropyl myristate, polysorbate 80 (Tween 80), polyoxyl 35 castor oil (cremophor castor oil), and polypropylene glycol (PG); or ethyl laurate, polysorbate 80 (Tween 80), polyoxyl 35 castor oil (cremophor castor oil), and polypropylene glycol (PG); or MCT, glycerol, polysorbate 80 (Tween 80), polyoxyl 35 castor oil (cremophor castor oil), polypropylene glycol (PG), and ethanol; or MCT, glycerol, polysorbate 80 (Tween 80), polyoxyl 35 castor oil (cremophor castor oil), polypropylene glycol (PG), ethanol, and at least one phospholipid; or MCT, glycerol, polysorbate 80 (Tween 80), polyoxyl 35 castor oil (cremophor castor oil), polypropylene glycol (PG), ethanol, transcutol, and at least one phospholipid; or MCT, glycerol, polysorbate 80 (Tween 80), polyoxyl 35 castor oil (cremophor castor oil), polypropylene glycol (PG), ethanol, oleic acid, and at least one phospholipid; or MCT, glycerol, polysorbate 80 (Tween 80), polyoxyl 35 castor oil (cremophor castor oil), polypropylene glycol (PG), ethanol, transcutol, oleic acid, and at least one phospholipid; or R (+)-limonene, polysorbate 80 (Tween 80), polypropylene glycol (PG), and ethanol; or castor oil, polysorbate 80 (Tween 80), Mirj S40, polypropylene glycol (PG), ethanol, and at least one phospholipid; or MCT, polysorbate 80 (Tween 80), polyoxyl 35 castor oil (cremophor castor oil), polypropylene glycol (PG), ethanol, oleic acid, and at least one phospholipid; or ethyl caprate, polysorbate 80 (Tween 80), polypropylene glycol (PG), ethanol, and at least one phospholipid; or—ethyl caprate, HECO 40, polyglyceryl-3 dioleate (CC497), polypropylene glycol (PG), acetic acid, and at least one phospholipid; or olive oil, Labrasol (oleoyl macrogolglycerides), polyglyceryl-3 dioleate (CC497), and ethanol; or olive oil, polysorbate 80 (Tween 80), polypropylene glycol (PG), ethanol, and at least one phospholipid; or MCT, polysorbate 80 (Tween 80), polyoxyl 35 castor oil (cremophor castor oil), polypropylene glycol (PG), and at least one phospholipid; or MCT, oleic acid, polysorbate 80 (Tween 80), polyoxyl 35 castor oil (cremophor castor oil), glycerol, polypropylene glycol (PG), ethanol, and at least one phospholipid; or Limonene, polysorbate 80 (Tween 80), polypropylene glycol (PG), and ethanol; or triacetin, polysorbate 80 (Tween 80), polypropylene glycol (PG), and at least one phospholipid; or triacetin, Labrasol (oleoyl macrogolglycerides), polyoxyl 35 castor oil (cremophor castor oil), polypropylene glycol (PG), isopropanol, and at least one phospholipid; or MCT, sucrose mono/dilaurate, polypropylene glycol (PG), isopropanol, and at least one phospholipid.

In some embodiments, the formulation may comprise per tablet or capsule 50-60 mg of medium-chain triglycerides or sesame oil, 480-515 mg of polyoxyl 35 castor oil (e.g., Cremophor EL), 110-125 mg of polysorbate 80 (e.g., Tween 80), 110-125 mg of PEG-8 caprylic/capric glycerides (oleoyl macrogolglycerides, e.g., Labrasol® ALF), 40-50 mg of polyglyceryl-3 oleate (e.g., Plurol® Oleique CC 947), 80-95 mg of propylene glycol, and/or 0.1-1 mg of butylated hydroxytoluene (BHT).

In some embodiments, the composition comprises per capsule or tablet: 45-55 mg of CBD, CBG, CBC, CBDV, or a combination thereof; 105-115 mg sesame oil; 375-385 mg of polyoxyl 35 castor oil; 275-285 mg of polysorbate 80; 135-145 mg of polyglyceryl-3 oleate; 25-35 mg of propylene glycol; 0.1-1 mg of butylated hydroxytoluene (BHT); or any combination thereof.

In some embodiments, the composition comprises per capsule or tablet: 95-105 mg of CBD, CBG, CBC, CBDV, or a combination thereof; 50-60 mg of medium-chain triglycerides; 480-490 mg of polyoxyl 35 castor oil; 110-120 mg of polysorbate 80; 110-120 mg of PEG-8 Caprylic/Capric Glycerides; 40-50 mg of polyglyceryl-3 oleate; 80-90 mg of propylene glycol; 0.1-1 mg of butylated hydroxytoluene (BHT); or any combination thereof.

In some embodiments the active ingredient solubilized by the formulation is a cannabinoid. In some embodiments, the active ingredient is a non-psychoactive cannabinoid. In some embodiments, the active ingredient is CBD or a CBD derivative.

In some embodiments, the pharmaceutical composition may be made by preparing the formulation via mixing and/or homogenizing the at least one oil, the at least one surfactant, and the at least one co-surfactant (and where applicable also at least one solvent, at least one co-solvent and/or at least one phospholipid), optionally while heating the mixture, adding at least one suitably pure cannabinoid to the formulation, mixing, or homogenizing the cannabinoid-formulation mixture until the cannabinoid is dissolved in the formulation mixture; and optionally, purifying, diluting, or further compounding the cannabinoid formulation.

In some embodiments, the concentrated (i.e., water-free) formulation is clear, transparent, and homogenous. In some embodiments, the diluted formulation is slightly opaque without visible particles or droplets.

In some embodiments, the liquid nanodomains remain completely homogeneous and almost monodispersed (i.e., the same size). In some embodiments, the liquid nanodomains range in size from 5 to 20 nm.

In some embodiments, the pH of both the concentrate and diluted formulations may be between 6.0 and 7.5.

In some embodiments, no physical changes are observed with storage of the formulation. In some embodiments, the active ingredient remains associated with the surfactant and lipid phases during storage in both concentrated and diluted forms.

In some embodiments, the formulation is chemically stable for at least 1 month, at least 3 months, at least 6 months, at least 1 year, at least 2 years, at least 3 years, or more than 3 years.

In some embodiments, the formulation is shelf stable at ambient conditions for at least 1 year, at least 2 years, at least 3 years, or more than 3 years.

In some embodiments the formulation contains less than 1 wt % water before any dilution. In some embodiments the formulation is contains less than 0.1 wt % water before any dilution. In some embodiments, the AIBEL formulation is water-free before any dilution.

### Pharmaceutical Compositions

Provided herein are pharmaceutical compositions comprising at least one cannabinoid solubilized in a pharmaceutically acceptable carrier (such as a formulation of the present disclosure).

In some embodiments, the pharmaceutical composition is chemically inert to the active compounds and has no detrimental side effects or toxicity under the conditions of use. In some embodiments, the choice of pharmaceutical carrier is determined in part by the active agent (e.g., cannabinoid), as well as by the method used to administer the composition.

In some embodiments, the pharmaceutical composition may comprise a variety of additional components, depending on the administration route and/or desired properties of the composition. In some embodiments, the pharmaceutical composition may comprise at least one additional component selected from, but not limited to, aqueous and non-aqueous diluents, isotonic sterile injection solutions, antioxidants, buffers, bacteriostats, suspending agents, solubilizers, thickening agents, gelling agent, emollients, moisturizers, stabilizers, preservatives, buffers, coloring agents, a fragrance, aromatic agents, flavoring agents, flavor masking agents, absorbers, filters, electrolytes, proteins, chelating agents, or combinations thereof.

In some embodiments, the pharmaceutical composition is in a form selected from a gel, a lotion, oil, soap, a spray, an emulsion, a cream, an ointment, capsules, soft gel capsules, chewing gum, a patch, buccal-patch and variety of other food products and supplements, or a solution.

In some embodiments, the pharmaceutical composition may be adapted for delivery of the active agent (e.g., cannabinoid) in one or more routes of administration. In some embodiments, the pharmaceutical composition may be adapted for delivery of the active agent (e.g., cannabinoid) in one or more routes of administration selected from, but not limited to, topical, buccal, oral, gavage, rectal, vaginal, transdermal, subcutaneous, intravenous, intramuscular, transdermal, intranasal, by inhalation, ocularly or parenterally into the circulatory system of a subject.

In some embodiments, the pharmaceutical composition is adapted for oral administration.

In some embodiments, the pharmaceutical composition suitable for oral administration may consist of (a) liquid solutions, such as an effective amount of the cannabinoid loaded formulation, optionally dissolved in diluents, such as water, saline, or juice (e.g. orange juice); (b) capsules, sachets, tablets, lozenges, and troches, each containing a predetermined amount of the cannabinoid, as solids or granules; (c) powders; (d) suspensions in an appropriate liquid; and/or (e) concentrates or diluted microemulsions (f) spray (g) inhalation. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, fluidizers (e.g., water) and com starch. Tablet forms can include one or more of lactose, sucrose, mannitol, com starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

In some embodiments, the pharmaceutical composition is administered in the form of a tablet, a capsule, a soft gel capsule, or a solution.

In some embodiments the pharmaceutical composition may be in the form of an 10-50 mg, 50-100 mg, 100-150 mg, 150-200 mg, 200-250 mg, 250-300 mg, 300-350 mg, 350-400 mg, 400-450 mg, 450-500 mg, 500-550 mg, 550-600 mg, 650-700 mg, 700-750 mg, 750-800 mg, 800-850 mg, 850-900 mg, 900-950 mg, 950-1000 mg, 1000-1050 mg, 1050-1100 mg, 1100-1150 mg, 1150-1200 mg, 1200-1250 mg, 1250-1300 mg, 1300-1350 mg, 1350-1400 mg, 1400-1450 mg, or 1450-1500 mg tablet or capsule. In some embodiments the pharmaceutical composition may be in the form of an 1500-1600 mg, 1600-1700 mg, 1700-1800 mg, 1800-1900 mg, or 1900-2000 mg capsule or tablet.

In some embodiments the embodiments the pharmaceutical composition may be in the form of an 1500 mg capsule or tablet. In some embodiments the embodiments the pharmaceutical composition may be in the form of an 1 mL capsule.

In some embodiments, the amount of pharmaceutically acceptable carriers or additional components can be selected as needed, for example, based on the desired rout of administration and the desired final form of the pharmaceutical composition. In some embodiments, the pharmaceutical composition is designed for oral delivery of a soft gel capsule and may contain between 34 wt % or 508 mg per capsule of said carriers or additional components.

In some embodiments, the pharmaceutical composition is formulated as a soft gel capsule and may comprise 10-150 mg of CBD, 50-60 mg of medium-chain triglycerides or sesame oil, 480-515 mg of polyoxyl 35 castor oil, 110-125 mg of polysorbate 80, 110-125 mg of PEG-8 Caprylic/Capric Glycerides, 40-50 mg of polyglyceryl-3 oleate, 80-95 mg of propylene glycol, 0.1-1 mg of butylated hydroxytoluene (BHT), 305-330 mg of gelatin, 130-150 mg of glycerin, 5-15 mg of caramel colorant, or any combination thereof, per capsule.

In some embodiments, the pharmaceutical composition is formulated as a soft gel capsule and may comprise 50 mg of synthetic CBD or CBD extracted from hemp, 57 mg of sesame oil, 511.1 mg of polyoxyl 35 castor oil (e.g., Cremophor EL), 121.6 mg of polysorbate 80 (e.g., Tween 80), 122.5 mg of PEG-8 Caprylic/Capric Glycerides, 47.5 mg of polyglyceryl-3 oleate (e.g., Plurol® Oleique CC 947), 90.25 mg of propylene glycol, 0.475 mg of butylated hydroxytoluene (BHT), 344 mg of gelatin, 152 mg of glycerin, 12 mg of caramel colorant, or any combination thereof, per capsule.

In some embodiments, the pharmaceutical composition is formulated as a soft gel capsule and may comprise 100 mg of synthetic CBD or CBD extracted from hemp, 54 mg of medium-chain triglycerides, 484.2 mg of polyoxyl 35 castor oil (e.g., Cremophor EL), 115.2 mg of polysorbate 80 (e.g., Tween 80), 116.1 mg of PEG-8 Caprylic/Capric Glycerides, 45 mg of polyglyceryl-3 oleate (e.g., Plurol® Oleique CC 947), 85.5 mg of propylene glycol, 0.45 mg of butylated hydroxytoluene (BHT), 344 mg of gelatin, 152 mg of glycerin, 12 mg of caramel colorant, or any combination thereof, per capsule.

In some embodiments, the pharmaceutical composition is formulated as a soft gel capsule and may comprise 45-55 mg of CBD, CBG, CBC, CBDV, or a combination thereof, 105-115 mg sesame oil, 375-385 mg of polyoxyl 35 castor oil, 275-285 mg of polysorbate 80, 135-145 mg of polyglyceryl-3 oleate, 25-35 mg of propylene glycol, 0.1-1 mg of butylated hydroxytoluene (BHT), or any combination thereof, per capsule. The capsule shell may comprise 340-350 mg of gelatin, 145-155 mg of glycerin, 5-15 mg of caramel colorant, and 30-40 mg water or any combination thereof, per capsule.

In some embodiments, the pharmaceutical composition is formulated as a soft gel capsule and may comprise 95-105 mg of CBD, CBG, CBC, CBDV, or a combination thereof, 50-60 mg of medium-chain triglycerides, 480-490 mg of polyoxyl 35 castor oil, 110-120 mg of polysorbate 80, 110-120 mg of PEG-8 Caprylic/Capric Glycerides, 40-50 mg of polyglyceryl-3 oleate, 80-90 mg of propylene glycol, 0.1-1 mg of butylated hydroxytoluene (BHT), or any combination thereof, per capsule. The capsule shell may comprise 340-350 mg of gelatin, 145-155 mg of glycerin, 5-15 mg of caramel colorant, 30-40 mg water, or any combination thereof, per capsule.

In some embodiments, the pharmaceutical composition is formulated as a soft gel capsule and may comprise 51 mg of synthetic CBD or CBD extracted from hemp, 110 mg of sesame oil, 380 mg of polyoxyl 35 castor oil, 280 mg of polysorbate 80, 140 mg of polyglyceryl-3 oleate, 30 mg of propylene glycol, 0.5 mg of butylated hydroxytoluene (BHT), or any combination thereof, per capsule. The capsule shell may comprise, 344 mg of gelatin, 152 mg of glycerin, 12 mg of caramel colorant, 33 mg of water, or any combination thereof, per capsule.

In some embodiments, the pharmaceutical composition is formulated as a soft gel capsule and may comprise 102 mg of synthetic CBD or CBD extracted from hemp, 54 mg of medium-chain triglycerides, 484 mg of polyoxyl 35 castor oil, 115 mg of polysorbate 80, 116 mg of PEG-8 Caprylic/Capric Glycerides, 45 mg of polyglyceryl-3 oleate, 85 mg of propylene glycol, 0.5 mg of butylated hydroxytoluene (BHT), or any combination thereof, per capsule. The capsule shell may comprise 344 mg of gelatin, 152 mg of glycerin, 12 mg of caramel colorant, 33 mg of water, or any combination thereof, per capsule.

In some embodiments, the pharmaceutical compositions can be produced by a process essentially comprising preparing the formulation via mixing and/or homogenizing the at least one oil, the at least one surfactant, and the at least one co-surfactant (and where applicable also at least one solvent, at least one co-solvent and/or at least one phospholipid), optionally while heating the mixture, adding at least one suitably pure cannabinoid to the formulation, mixing, or homogenizing the cannabinoid-formulation solution until the cannabinoid is dissolved in the formulation; and optionally, purifying, or diluting, the cannabinoid formulation. Said cannabinoid formulation is then optionally further combined with pharmaceutically acceptable carriers or additional components through such methods as are known in the art to produce the desired final pharmaceutical formulation.

### Dosing and Administration

Social Anxiety Disorder (SAD) is an early onset chronic disorder that untreated results in high chronicity, comorbidity, health care utilization, and associated functional and quality of life impairment. Standard treatments are cognitive behavioral therapy (CBT) and various forms of pharmacotherapy, both of which are only moderately effective. First line pharmacotherapy for daily, long-term reduction of SAD symptoms include antidepressants, particularly selective serotonin- and serotonin-noradrenaline reuptake inhibitors (SSRI/SNRIs). Few if any medications that target mechanisms for treating SAD have progressed to clinical use over the last 30 years, despite several promising available targets from translational studies of neurocircuits underlying fear regulation.

The present disclosure presents methods for the treatment of anxiety disorder, e.g., generalized anxiety disorder, panic disorder, social anxiety disorder, phobia-related disorder (e.g., aerophobia, acrophobia, zoophobia, trypanophobia, hemophobia, agoraphobia, or the like), separation anxiety disorder, selective mutism, medication-induced anxiety disorder, or the like, and symptoms thereof, as well as methods of manufacturing said compositions, and kits useful in the practice of the present disclosure.

Rodent models and clinical neuroimaging studies have shown many anxiety disorder symptoms as being linked to a dysfunction in frontotemporal circuits. Normally, adaptive regulation of the neuroendocrine and autonomic response and fear associated with traumatic experiences depends on coordinated activity between the prefrontal (and cingulate) cortex (PFC) and hippocampus (HPC), leading to top-down inhibition of extended amygdala activity. In anxiety disorder, this PFC-HPC-mediated top-down inhibition is impaired. Executive function, emotion regulation and other key neurocognitive impairments in anxiety disorder also depend on PFC and HPC functional integrity. Therefore, medications that enhance the capacity of PFC-HPC circuits to regulate of stress- or fear associated amygdala activity may improve relevant symptom domains in anxiety disorder.

The present disclosure presents pharmaceutical compositions and methods of their use in the treatment of anxiety disorders, e.g., social anxiety disorder. In some embodiments, pharmaceutical compositions comprise at least one cannabinoid and at least one pharmaceutically acceptable carrier (e.g., formulation). In some embodiments, the pharmaceutical compositions comprise at least one additional component to aid in administration of the pharmaceutical composition.

The present disclosure presents methods for the effective treatment of anxiety disorder, or the lessening of anxiety disorder associated symptoms in a subject in need thereof. The present disclosure presents methods for the effective treatment of anxiety disorder, or the lessening of anxiety disorder associated symptoms in a subject, by administering to the subject a cannabinoid (e.g., CBD) containing pharmaceutical composition on a prescribed schedule, according to the present disclosure.

To effectively gauge successful treatment with the pharmaceutical composition of the disclosure, the presence and severity of anxiety disorders can be established via several methods that are well known in the art. For example, the clinician administered Structured Clinical Interview Schedule for DSM-5 (SCID-5) score can be administered and scored, to determine the presence of social anxiety disorder, in which the Liebowitz Social Anxiety Scale (LSAB) may indicate increasing severity of symptoms when a patient has a score of at least 60.

In some embodiments, the Structured Clinical Interview Schedule for DSM-5 (SCID-5) score in a subject may be reduced by the administration of the pharmaceutical composition, as compared to the subject's pre-treatment score.

In some embodiments, the Structured Clinical Interview Schedule for DSM-5 (SCID-5) may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, the Liebowitz Social Anxiety Scale (LSAS) score for social anxiety is reduced by the administration of the pharmaceutical composition, as compared to the subject's pre-treatment score.

In some embodiments, the Liebowitz Social Anxiety Scale (LSAS) score for social anxiety may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, the Liebowitz Social Anxiety Scale (LSAS) score for social anxiety may be reduced by 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 points by the administration of the pharmaceutical composition, as compared to the subject's pre-treatment score.

In some embodiments, the Liebowitz Social Anxiety Scale (LSAS) score for social anxiety severity rating may drop by 1, 2, 3, or 4 levels (e.g., from extreme to severe, from extreme to moderate, from extreme to mild, from severe to moderate, from severe to mild, from moderate to mild, or from any level to absent) by the administration of the pharmaceutical composition, as relative to the pre-treatment severity.

In some embodiments, the General Anxiety Disorder (GAD-7) score for a subject may be used to determine severity of overall anxiety in a subject suffering from anxiety disorders. Reduction in this score would likewise indicate a therapeutic benefit of a treatment.

In some embodiments, the GAD-7 may be reduced by the administration of the pharmaceutical composition, as compared to the subject's pre-treatment score.

In some embodiments, the GAD-7 score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, the GAD-7 score may be reduced by 21, 20, 19, 18, 17, 16, 15, 14, 13, 12, 11, 10, 9, 8, 7, 6, 5, 4, 3, 2, or 1 points by the administration of the pharmaceutical composition, as compared to the subject's pre-treatment score.

In some embodiments, GAD-7 (Anxiety) severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the Trier Social Stress Test (TSST) score for a subject may be used to determine severity of overall anxiety in a subject suffering from anxiety disorders. Reduction in this score would likewise indicate a therapeutic benefit of a treatment.

In some embodiments, the TSST may be reduced by the administration of the pharmaceutical composition, as compared to the subject's pre-treatment score.

In some embodiments, the TSST score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, TSST severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the Clinical Global Impression of Severity and Improvement (CGI-I and S) score for a subject may be used to determine severity of overall anxiety in a subject suffering from anxiety disorders. Reduction in this score would likewise indicate a therapeutic benefit of a treatment.

In some embodiments, the CGI-I and S may be reduced by the administration of the pharmaceutical composition, as compared to the subject's pre-treatment score.

In some embodiments, the CGI-I and S score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, CGI-I and S severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the Quick Inventory of Depressive Symptomology, Self-Report (QIDS-SR) score for a subject may be used to determine severity of overall anxiety in a subject suffering from anxiety disorders. Reduction in this score would likewise indicate a therapeutic benefit of a treatment.

In some embodiments, the QIDS-SR may be reduced by the administration of the pharmaceutical composition, as compared to the subject's pre-treatment score.

In some embodiments, the QIDS-SR score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, QIDS-SR severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the Perceived Stress Scale (PSS-14) score for a subject may be used to determine severity of overall anxiety in a subject suffering from anxiety disorders. Reduction in this score would likewise indicate a therapeutic benefit of a treatment.

In some embodiments, the PSS-14 may be reduced by the administration of the pharmaceutical composition, as compared to the subject's pre-treatment score.

In some embodiments, the PSS-14 score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, PSS-14 severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the Anxiety Sensitivity Index-3 (ASI-3) score for a subject may be used to determine severity of overall anxiety in a subject suffering from anxiety disorders. Reduction in this score would likewise indicate a therapeutic benefit of a treatment.

In some embodiments, the ASI-3 may be reduced by the administration of the pharmaceutical composition, as compared to the subject's pre-treatment score.

In some embodiments, the ASI-3 score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, ASI-3 severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the insomnia severity index score for a subject may be used to determine severity of overall anxiety in a subject suffering from anxiety disorders. Reduction in this score would likewise indicate a therapeutic benefit of a treatment.

In some embodiments, the insomnia severity index may be reduced by the administration of the pharmaceutical composition, as compared to the subject's pre-treatment score.

In some embodiments, the insomnia severity index score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, insomnia severity index severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the PROMIS Anxiety score for a subject may be used to determine severity of overall anxiety in a subject suffering from anxiety disorders. Reduction in this score would likewise indicate a therapeutic benefit of a treatment.

In some embodiments, the PROMIS Anxiety score may be reduced by the administration of the pharmaceutical composition, as compared to the subject's pre-treatment score.

In some embodiments, the PROMIS Anxiety score may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, PROMIS Anxiety severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments, the PROMIS emotional distress score, Altman Self-Rating Mania Scale (ASRM) score, Patient Health Questionnaire 15 Somatic Symptom Severity Scale (PHQ-15) score, PROMIS Sleep Disturbance score, Florida Obsessive Compulsive Inventory (FOCI) Severity Scale (Part B) score, Substance Use score, Patient Health Questionnaire (PHQ-9) score, Severity Measure for Separation Anxiety Disorder score, Severity Measure for Specific Phobia score, Severity Measure for Panic Disorder score, Severity Measure for Agoraphobia score, or Brief Dissociative Experiences Scale (DES-B) for a subject may be used to determine severity of overall anxiety in a subject suffering from anxiety disorders. Reduction in this score would likewise indicate a therapeutic benefit of a treatment.

In some embodiments, the ASRM score, PHQ-15 score, PROMIS Sleep Disturbance score, FOCI-Part B score, Substance Use score, PHQ-9 score, Severity Measure for Separation Anxiety Disorder score, Severity Measure for Specific Phobia score, Severity Measure for Panic Disorder score, Severity Measure for Agoraphobia score, or DES-B may be reduced by the administration of the pharmaceutical composition, as compared to the subject's pre-treatment score.

In some embodiments, the ASRM score, PHQ-15 score, PROMIS Sleep Disturbance score, FOCI-Part B score, Substance Use score, PHQ-9 score, Severity Measure for Separation Anxiety Disorder score, Severity Measure for Specific Phobia score, Severity Measure for Panic Disorder score, Severity Measure for Agoraphobia score, or DES-B may be reduced by 100%, 95%, 90%, 85%, 80%, 75%, 70%, 65%, 60%, 55%, 50%, 45%, 40%, 35%, 30%, 25%, 20%, 15%, 10%, or 5% by the administration of the pharmaceutical composition, as compared to the subject's pre-treatment score.

In some embodiments, ASRM, PHQ-15, PROMIS Sleep Disturbance, FOCI-Part B, Substance Use, PHQ-9, Severity Measure for Separation Anxiety Disorder, Severity Measure for Specific Phobia, Severity Measure for Panic Disorder, Severity Measure for Agoraphobia, or DES-B severity rating may drop by 1, 2, 3, or 4 levels (e.g., from severe to moderate, from severe to mild, from severe to minimal, from moderate to mild, from moderate to minimal, from mild to minimal or from any level to absent) by the administration of the pharmaceutical composition, as relative to the pre-treatment severity.

In some embodiments the method for treating anxiety disorders, e.g., social anxiety disorder, is comprised of administering a pharmaceutical composition comprising at least, one or more cannabinoids and one or more formulations to a subject.

In some embodiments, the at least one cannabinoid is a non-psychoactive cannabinoid. In some embodiments, said cannabinoid is CBD or a CBD derivative.

In some embodiments the total amount of the at least one cannabinoid (e.g., CBD) administered each day (total daily dose) is selected from but not limited to between 50 mg/day and 2000 mg/day, between 100 mg/day and 2000 mg/day, between 200 mg/day and 1400 mg/day, between 200 mg/day and 600 mg/day, between 700 mg/day and 1400 mg/day. Example doses include but are not limited to 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day, or 1000 mg/day, 1400 mg/day, 1500 mg/day, 1750 mg/day, or 2000 mg/day.

In some embodiments, the at least one cannabinoid (e.g., CBD) is administered at a daily dose of at least 10 mg/day.

In some embodiments, the total daily dose of the at least one cannabinoid is 400 mg/day. In some embodiments, the total daily dose of the at least one cannabinoid is 600 mg/day to 2000 mg/day.

In some embodiments, the pharmaceutical composition is administered once a day. In some embodiments, the pharmaceutical composition is administered twice a day. In some embodiments, the pharmaceutical composition is administered more than twice a day.

In some embodiments, the total amount of cannabinoid administered a day (total daily dose) is administered in a single daily dose.

In some embodiments, the total amount of cannabinoid is administered over the course of the day in multiple smaller doses that additively equal the total daily dose (a split daily dose). In some embodiments, all split daily doses are equivalent in amount of cannabinoid present. In some embodiments, the amount of cannabinoid present varies in each split daily dose.

In some embodiments, the total daily dose of at least one cannabinoid administered each day may change over the course of treatment. In some embodiments, the total daily dose of at least cannabinoid administered each day may decrease over the course of treatment. In some embodiments, the total daily dose of at least one cannabinoid administered each day may increase over the course of treatment.

In some embodiments the total daily dose of cannabinoid may increase after one week of treatment. In some embodiments, the total daily dose of at least one cannabinoid may increase from 200 mg/day to 400 mg/day over the course of treatment. In some embodiments, the total daily dose of at least one cannabinoid may increase from 200 mg/day to 400 mg/day after one week of treatment. In some embodiments, the total daily dose of at least one cannabinoid may increase from 200 mg/day to 400 mg/day after two weeks of treatment.

In some embodiments, the total daily dose of at least one cannabinoid may increase from 200 mg/day to 400 mg/day over the course of treatment. In some embodiments, the total daily dose of at least one cannabinoid may increase from 200 mg/day to 400 mg/day after one week of treatment. In some embodiments, the total daily dose of at least one cannabinoid may increase from 400 mg/day to 600 mg/day after two weeks of treatment.

In some embodiments, the total daily dose of at least one cannabinoid may change at the discretion of an attending appropriately licensed medical practitioner over the course of treatment.

In some embodiments the pharmaceutical composition in the form of a tablet, a capsule, a soft gel capsule, or a solution.

In some embodiments the pharmaceutical composition is administered orally.

In some embodiments the pharmaceutical composition is in the form of a soft gel capsule for administration orally as a split daily dose (2 doses per day) and provides a total daily dose of CBD of 600 mg/day.

In some embodiments the pharmaceutical composition is in the form of a soft gel capsule for administration orally as a split daily dose (2 doses per day) and provides a total daily dose of CBD of 400 mg/day.

In some embodiments, kits may be provided to perform the disclosure, said kits comprising a pharmaceutical composition of the disclosure and instructions for carrying out the methods of the disclosure. In some embodiments, said pharmaceutical composition may be supplied in white HDPE bottles with child-resistant HDPE bottle caps. In some embodiments, the kits may be packaged and labeled in compliance with the Good Manufacturing Practice for drugs used in clinical trials. In some embodiments, the instructions may be provided electronically, via data-storage device, or in paper format.

In some embodiments a probability of response (POR) to treatment with the disclosed methods or the disclosed pharmaceutical compositions may be produced via the method essentially comprised of collecting a subject's demographic, clinical (including medication and substance use information), and biological (i.e., genetic and other blood-based markers) information as well as task-based and structural magnetic resonance neuroimaging markers centered around hippocampal, prefrontal and extended amygdala function and fear extinction before administration of the pharmaceutical composition or placebo; administering the disclosed pharmaceutical composition or placebo and collecting the measures again after administration, and applying a random forest algorithm to generate a POR for each subject.

In some embodiments, the selection of subjects most likely to benefit from treatment with the disclosed methods or the disclosed pharmaceutical compositions may be carried out via the method comprised essentially of, a) collecting a subject's demographic, clinical (including medication and substance use information), and biological (i.e., genetic and other blood-based markers) information as well as task-based and structural magnetic resonance neuroimaging markers centered around hippocampal, prefrontal and extended amygdala function and fear extinction, b) applying a probability classifier to the features collected to predict the POR to the disclosed pharmaceutical composition for the subject, c) selecting subjects who have a predicted POR greater than a determined threshold (e.g., POR >0.6).

### EQUIVALENTS AND SCOPE

Those skilled in the art will recognize or be able to ascertain using no more than routine experimentation, many equivalents to the specific embodiments in accordance with the present disclosure described herein. The scope of the present disclosure is not intended to be limited to the above Description.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The term “comprising” is intended to be open and permits but does not require the inclusion of additional elements or steps. When the term “comprising” is used herein, the term “consisting of” is thus also encompassed and disclosed.

Where ranges are given (e.g., 1-5, or 2 to 10, or between 7 and 12), endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

It is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1. Pharmaceutical Compositions Useful for Practicing the Disclosure

All inactive ingredients included in the below formulations have been previously approved by the FDA for use as excipients in oral medications or food additives.

For all compositions listed below, CBD was obtained via extraction from hemp by Mile High Labs (Broomfield, CO, USA). CBD+excipient formulation and encapsulation were performed by Baxco Pharmaceutical (Irwindale, CA, USA). In short, excipients were emulsified, then CBD added, then the mixture was re-emulsified and encapsulated using standard commercial encapsulation techniques.

### Composition A and Composition A′

The list of components and their amounts per 1500 mg (1 mL) capsule for the Composition A and Composition A′ pharmaceutical compositions are given in Table 1.

TABLE 1
Pharmaceutical Composition A and A′
FOrmulation Composition

| | | Amount | |
|---|---|---|---|
| | | per | |
| | Component | Capsule | wt % |
| Component | Type | (mg) | (Formulation) |
| Cannabidiol (CBD) | Cannabinoid | 51 | 5 |
| Polyoxyl 35 Castor Oil | Hydrophilic | 380 | 38 |
| | Surfactant | | |
| Polysorbate 80 | Hydrophilic | 280 | 28 |
| | Surfactant | | |
| Polyglyceryl-3 Oleate | Co-Surfactant | 140 | 14 |
| Propylene Glycol | Co-Surfactant | 30 | 3 |
| Sesame Oil | Oil | 110 | 11 |
| Butylated Hydroxytoluene | Additive | 0.5 | 0.05 |
| (BHT) | | | |

Softgel Capsule Shell

| | | Amount | |
|---|---|---|---|
| | | per | |
| | Component | Capsule | wt % |
| Component | Type | (mg) | (Shell) |
| Gelatin | Shell Material | 344 | 64 |
| Glycerin | Plasticizer | 152 | 28 |
| Caramel Color | Colorant | 12 | 2 |
| Purified Water (After | Fluidizer | 33 | 6 |
| Drying) | | | |

For administration of the Composition A, these capsules are subsequently broken open and then further diluted 50/1 with water.

The chemical stability of the Composition A was evaluated at 25° C. and 40° C. for three months. No change in assay or impurities was detected. The examination of the physical stability of the Composition A was conducted using the LUMiFuge™ analytical centrifugation for rapid and efficient measurement, enabling prediction of physical stability and shelf life of a product. The liquid nanodomains of the formulation were shown to be stable a 3K rpm for over 17 hours, conditions equivalent to 2 years of storage.

Several tests were conducted to determine the chemical characteristics of composition A′, both in concentrated and diluted forms. The concentrated (i.e., water free) formulation was clear, transparent, and homogeneous. The diluted formulation is slightly opaque without visible particles or droplets and the nanometric droplets remain completely homogeneous and almost monodispersed (i.ee, the same size). All liquid nanodomain droplets range in size from 5 to 20 nm. The pH of both the concentrate and diluted formulations is between 6.0 and 7.5. No physical changes were observed with storage and CBD had a Log P of 6 and remained associated with the surfactant and lipid phases during storage in both concentrated and diluted forms.

### Composition B

The list of components and their amounts per 1500 mg (1 mL) capsule for Composition Bis given in Table 2.

TABLE 2
Pharmaceutical Composition B
Formulation Composition

| | | Amount per | wt % |
|---|---|---|---|
| Component | Component Type | Capsule (mg) | (Formulation) |
| Cannabidiol | Cannabinoid | 102 | 10 |
| Polyoxyl 35 Castor Oil | Hydrophilic Surfactant | 484 | 48.4 |
| Polysorbate 80 | Hydrophilic Surfactant | 115 | 11.51 |
| Polyglyceryl-3 Oleate | Co-Surfactant | 45 | 4.5 |
| Propylene Glycol | Co-Surfactant | 85 | 8.55 |
| PEG-8 caprylic/capric | Hydrophilic Surfactant | 116 | 11.6 |
| glycerides (Labrasol ® ALF) | | | |
| Medium Chain Triglycerides | Oil | 54 | 5.4 |
| Butylated Hydroxytoluene | Additive | 0.5 | 0.05 |
| (BHT) | | | |

Softgel Capsule Shell

| | | Amount per | wt % |
|---|---|---|---|
| Component | Component Type | Capsule (mg) | (Shell) |
| Gelatin | Shell Material | 344 | 64 |
| Glycerin | Plasticizer | 152 | 28 |
| Caramel Color | Colorant | 12 | 2 |
| Purified Water (After Drying) | Fluidizer | 33 | 6 |

To characterize physical and chemical characteristics of the formulation visual and microscopic examinations were made of both the concentrated and 99 wt % water diluted forms to demonstrate single-phase, transparent attributes, compatibility with Softgel and their components was tested, Dynamic Light Scattering (DLS) measurements were made to determine the nano-droplets average size, and LUMisizer® analysis was performed to assess physical stability of the nanodomains.

The Composition B formulation showed similar or better dilutability than the Composition A or Composition A′ formulations with no particle precipitation or oil-droplet formation. Encapsulation within gelatin soft gels was found to be feasible with no deformation of the capsule's shell. The Composition B formulation was found to be stable and predicted shelf-stable under ambient conditions for 2.3 years. DLS determined the formulation to be mono dispersed with a relatively low PDI (0.020-0.300) and a single detected population of 20 nm.

### Example 2: Pharmacokinetics

### Composition A Formulation in Rat

Sprague Dawley male rats (average weight 250 g) received a single dose of 8 mg/kg (i.e., an average of 2.0 mg/animal) via gavage feeding. The CBD concentration in all formulations was 4.0 mg/mL. Blood samples for CBD plasma concentrations were collected at 0.25, 0.5, 2.0, 4.0, 8.0 and 12 hrs after dosing. Six different formulations were evaluated and compared to a Control formulation (CBD in olive oil) at the same concentration. There were 5 animals in each group. Of the six formulations, two were selected, due to their unique PK parameters, as candidates for a preliminary human PK study:

### Composition A and Composition C.

Table 3 outlines the average concentrations and the ratio between the Composition A formulation and the Control formulation at each time point.

TABLE 3
Average CBD Plasma Concentrations per Time Point and IP/Control Ratio

| Formulation | Time (HR) | 0.25 | 0.5 | 2.0 | 4.0 | 8.0 | 12.0 |
|---|---|---|---|---|---|---|---|
| Composition | Ave (ng/mL) | 28.10 | 137.34 | 213.77 | 54.65 | 18.66 | 5.40 |
| A (N = 5) | [SD]* | [0.76] | [5.49] | [16.82] | [4.42] | [3.58] | [0.58] |
| | Ratio | 3.86 | 2.90 | 1.62 | 1.69 | 3.13 | 2.08 |
| Control | Ave (ng/mL) | 7.28 | 47.43 | 131.68 | 32.29 | 5.97 | 2.60 |
| | [SD]* | [0.83] | [3.40] | [4.76] | [1.90] | [0.91] | [0.09] |

*Ave: average, [SD]: Standard deviation
**Ratio: IP/Control

The PK parameters (Tₘₐₓ, Cₘₐₓ and AUC₀₋₁₂) for the Composition A formulation are summarized in Table 4. The tested formulation shows advantages in either Cₘₐₓ, AUC₀₋₁₂ and/or Tₘₐₓ values compared to the Control formulation.

TABLE 4
CBD Rat PK Parameters and Ratio of IP vs. Control

| Formulation | | Tₘₐₓ (hr) | Cₘₐₓ (ng/mL) | AUC₀₋₁₂ (ng*h/mL) |
|---|---|---|---|---|
| Composition A | Ave* | 2.0 | 213.77 | 750.65 |
| | [SD] | 0.0 | 16.82 | 41.65 |
| | Ratio** | 1.0 | 1.62 | 1.88 |
| | % Increase | 0% | 62.34% | 87.80% |
| Control | Ave* | 2.0 | 131.68 | 399.70 |
| | [SD] | 0.0 | 4.76 | 11.46 |

*Ave: average, [SD]: Standard deviation
**Ratio: Composition A/Control

Composition A was selected for the preliminary human PK trial because its bioavailability parameters (Cₘₐₓ and AUC₀₋₁₂) were markedly superior to the Control formulation. Tₘₐₓ, however, was the same (2 hrs). This is significant when comparing the Composition A formulation with the known published data of the commercialized and FDA approved Epidiolex® product in which the Tₘₐₓ was measured between 4 to 5 hours post oral administration.

### Composition B Formulation in Rat

The Composition B formulation was evaluated in a PK study in rats. The PK parameters (Tₘₐₓ, Cₘₐₓ and AUC_{0-∞}) are summarized in Table 5. The formulation was evaluated versus the Composition A formulation and a comparison of fasted and fed dosing was conducted. The fed rats exhibited somewhat higher Cₘₐₓ values compared to fasting rats for the Composition B formulation (205 ng/mL fed versus 170 ng/ml fasted) but the Composition A formulation showed a lower Cₘₐₓ value (37 ng/ml fed versus 51 ng/mL fasted). The fed condition, however, resulted in shortened Tₘₐₓ values for both the Composition B and the Composition A formulations, indicating faster absorption. For the Composition B formulation, the Tₘₐₓ for the fed state was 0.5 hr compared with 3.0 hr for the fasted state.

TABLE 5
CBD Rat PK Parameters and Ratio of IP vs. Control

| Formulation | | Tₘₐₓ (hr) | Cₘₐₓ (ng/mL) | AUC_{0-∞} (ng*h/mL) |
|---|---|---|---|---|
| Composition B | Ave* | 3.0 | 170 | 1211 |
| | Ratio* | 1.00 | 1.67 | 2.18 |
| | % Increase | 0% | 67% | 118% |
| Composition A | Ave* | 3.0 | 102 | 556 |
| (reference) | | | | |

*Ave: average
**Ratio: Composition B/Composition A

In terms of bioavailability, the Composition B formulation was superior to the Composition A formulation, with an increase in Cₘₐₓ in the fasted condition of 67% (170 ng/mL vs 102 ng/mL) and an increase in AUC of 120% (1211 ng·hr/mL vs 556 ng·hr/mL) on a dose adjusted basis.

### Composition A in Humans

In this study, fifteen subjects were dosed at 50 mg of CBD and then monitored for safety and pharmacokinetics using the Composition A excipient nanodomain formulation with CBD in olive oil as a reference. The CBD used for this study was synthetically produced. The subjects were dosed with a 1 mL formulation that was diluted with water to a 2% concentration and then taken orally. The study design was a single-dose, crossover study with a 7-day washout period between doses. All dosing was conducted in a fasted state (10 hr min). A summary of the PK data from this study is noted in Table 6.

TABLE 6
Oral Bioavailability Parameters from Healthy Volunteer Studies for the Composition A Formulation

| | Dose | Statistical | Tₘₐₓ | Cₘₐₓ | AUC₀₋₂₄ | AUC_{0-∞} | T_{1/2} |
|---|---|---|---|---|---|---|---|
| Compound | (mg) | Variable | (hrs)¹ | (ng/mL)² | (ng*h/mL)² | (ng*h/mL)² | (hrs) |
| Composition A | 50 | Mean | 0.97 | 14.58 | 37.14 | 38.62 | 3.33 |
| | | (SD) | (0.05) | (4.79) | (16.26) | (16.62) | (0.96) |
| | | SEM | 0.133 | 1.279 | 4.345 | 4.441 | 0.255 |
| | | Median | 1.00 | 14.55 | 37.00 | 38.45 | 3.75 |
| | | (range) | (0.50-2.00) | (7.30-24.36) | (18.80-80.54) | (19.72-82.99) | (1.99-4.54) |
| CBD in Oil | 50 | Mean | 1.72 | 13.05 | 36.00 | 37.49 | 3.27 |
| (control) | | (SD) | (0.83) | (10.83) | (26.26) | (26.48) | (0.91) |
| | | SEM | 0.221 | 2.893 | 7.018 | 7.076 | 0.243 |
| | | Median | 1.75 | 9.79 | 26.48 | 27.92 | 3.22 |
| | | (range) | (0.50-4.00) | (4.57-47.27) | (12.49-116.10) | (13.57-117.99) | (2.26-4.35) |
| % Change | 0% | Mean | −43.60% | 11.72% | 3.17% | 2.99% | 1.83% |

### Example 3: Clinical Trial

### Study Design

A double-blind randomized placebo-controlled study may be completed to study the effect of oral CBDs on anxiety disorder symptoms and neurocognitive function in 120 social anxiety disorder diagnosed patients. CBD vs placebo administration may occur daily over eight weeks, with three Treatment Arms: Arm 1 (CBD 800 mg/day), Arm 2 (CBD 400 mg/day), and Arm 3 (Placebo (PBO)). CBD dose is titrated from 200 mg to the final dose over the first 2 weeks. The Primary outcome, social anxiety symptoms, and related secondary outcomes may be assessed at baseline, and weekly throughout the trial. Neurocognitive function is assessed at baseline and week 8 following treatment. This study employs an adaptive dose finding design, as follows. Following recruitment of half the intended subjects n=60, an independent statistician (not part of the study team) conducts a comparison of the two active study doses according to a priori specified criteria, and the remainder of the trial proceeds as a two-arm study. Plasma CBD levels may be assessed at select time points over eight weeks to obtain partial PK and assess for dose-accumulation. THC levels may be measured to assess whether conversion from CBD to THC occurs.

### Exclusion Criteria

Due to potential interactions with CBD that are likely to decrease the effectiveness or safety of treatment, potential subjects may be excluded from treatment if they demonstrate any of the following criteria: have a lifetime history of bipolar disorder, schizophrenia, psychosis, or delusional disorders, obsessive-compulsive disorders or an eating disorders in the past 12 months, neurocognitive disorders, intellectual disabilities, communication disorders or other cognitive dysfunction that could interfere with capacity to engage in therapy or complete study procedures, major depressive disorder, or substance or alcohol use disorder (other than nicotine) in the last 6 months; have a positive urine toxicology for illicit drugs and/or cannabinoids, or self-reported use of CBD, THC or marijuana in the past 4 weeks prior to baseline; have a significant suicidal ideation (assessed by CSSRS SI score greater than 2) or who have enacted suicidal behaviors within 6 months prior to intake; have not been free of concurrent psychotropic medication including those acting on serotonergic pathways, antidepressants, antipsychotics, anticonvulsants, benzodiazepines, and psychostimulants, treatments for addictions, and medications metabolized primarily by CYP3A4, CYP3A5, or CYP2D6 enzymes that also have a narrow therapeutic index for at least 4 weeks prior to initiation of randomized treatment; have an inability to understand study procedures or informed consent process, or significant personality dysfunction likely to interfere with study participation (assessed during the clinical interview) or inability to comply with study procedures (such as planned extended travel) assessed on clinical interview; have a current serious unstable medical illness, or a condition for which hospitalization may be likely within the next year as assessed by medical history and physical exam; have a clinically significant abnormal physical examination, vital signs or 12 lead ECG at screening, clinically significant abnormal value for hematology, clinical chemistry, or urinalysis at screening, including elevated levels of transaminases (ALT and/or AST); are pregnant or of child bearing potential who are not using medically accepted forms of contraception; have any concurrent psychotherapy initiated within 3 months of baseline, or ongoing psychotherapy of any duration directed specifically toward treatment of SAD; have received an investigational drug or used an invasive investigational medical device within 1 month or within a period less than 10 times the drug's half-life, whichever is longer, before Day 1; have a history of head trauma causing loss of consciousness, seizure or ongoing cognitive impairment; have contraindications for MRI including metal implants, surgical clips, probability of metal fragments, or braces that are prohibited; or are left-handed.

### Study Drug

This study utilizes Composition A′ described in Example 1. The composition of the placebo Softgel capsule is given in Table 7.

TABLE 7
Placebo Composition
Formulation Composition

| | | Amount | |
|---|---|---|---|
| | | per | |
| | Component | Capsule | wt % |
| Component | Type | (mg) | (Formulation) |
| Cannabidiol | Cannabinoid | 0 | 0 |
| Polyoxyl 35 Castor Oil | Hydrophilic | 405 | 40.5 |
| | Surfactant | | |
| Polysorbate 80 | Hydrophilic | 290 | 29 |
| | Surfactant | | |
| Polyglyceryl-3 Oleate | Co-Surfactant | 150 | 15 |
| Propylene Glycol | Co-Surfactant | 35 | 3.5 |
| Sesame Oil | Oil | 120 | 12 |
| Butylated Hydroxytoluene | Additive | 0.5 | 0.05 |
| (BHT) | | | |

Softgel Capsule Shell

| | | Amount | |
|---|---|---|---|
| | | per | |
| | Component | Capsule | wt % |
| Component | Type | (mg) | (Shell) |
| Gelatin | Shell Material | 344 | 64 |
| Glycerin | Plasticizer | 152 | 28 |
| Caramel Color | Colorant | 12 | 2 |
| Purified Water (After | Fluidizer | 33 | 6 |
| Drying) | | | |

### Dosing and Administration

Participants may be divided into 3 study Arms: Arm 1, the high dose group, Arm 2, the low dose group, and Arm 3, placebo. All groups receive the same number of capsules each day and at approximately the same time. Capsules are taken orally.

The duration of the active treatment portion of the study is 8 weeks, plus a follow-up phone call (post-treatment) at 9 weeks. For participants to be included in the primary statistical analyses, they must complete the 8-week treatment phase of the study.

### Dose Schedule Arm 1

For the first week, participants in Arm 1, may be administered eight 50 mg CBD capsules every morning following a light meal (total daily dose of 400 mg CBD). For the second week, Arm 1 continues to receive eight 50 mg CBD capsules each morning and an additional eight 50 mg CBD capsules with their evening meal (total daily dose of 400 mg CBD). For the following 6 weeks the morning regimen remains the same and participants in Arm 1 receive an additional eight 50 mg CBD capsules each evening (total daily dose of 800 mg CBD).

### Dose Schedule Arm 2

For the first week, participants in Arm 2, may be administered four 50 mg CBD capsules every morning following a light meal an additional four placebo capsules with their evening meal (total daily dose of 200 mg CBD). For the second week, Arm 2 continues to receive four 50 mg CBD capsules each morning and an additional four placebo capsules with their evening meal (total daily dose of 200 mg CBD). For the following 6 weeks the morning regimen remains the same, and participants in Arm 2 receive an additional four 50 mg CBD capsules each evening (total daily dose of 400 mg CBD).

### Dose Schedule Arm 3

Arm 3 receives the same number of capsules at the same time as the other 2 study arms, but all capsules are placebo (0 mg CBD).

### Analysis

PK Factors: Blood plasma (8.5 mL) may be collected at six time points. Plasma cannabinoid levels of CBD and THC (none expected) is obtained 30 min prior to, and 60 min following administration of study drug on day 1 (D1), Week 2 (D4) and Week 8 (D10) of treatment. To appraise pharmacokinetics, mixed-model for repeated measure (MMRM) may be used to compare change over two hours and over two weeks in plasma levels between the two doses of CBD to each other. Although no levels are expected, CBD may be compared to placebo as well to verify cannabinoid prior abstinence.

Psychiatric Factors: Social Anxiety diagnosis and severity are also assessed at screening and at week 8 with the Clinician Administered Structured Clinical Interview Schedule for DSM-5 (SCID-5), Trier Social Stress Test (TSST), and Liebowitz Social Anxiety Scale (LSAS) for the DSM-5. Social Anxiety disorder symptoms are assessed with the Social Anxiety Disorder Checklist for the DSM-5. Symptoms are assessed with the PROMIS Anxiety Score and Insomnia Severity Index score. Mood and anxiety symptoms are measured with the General Anxiety Disorder 7 Item Scale (GAD-7) the Anxiety Sensitivity Index-3 (ASI-3), the Perceived Stress Scale (PSS-14), the Quick Inventory of Depressive Symptomology, Self-Report (QIDS-SR), and the Clinical Global Impression of Severity and Improvement (CGI-I and S).

Baseline Patient Psychiatric and Other History Measures: A battery of clinician and self-report instruments may be administered at baseline to obtain comprehensive information for patient-centered psychiatric, emotional, and physical trauma.

Fear conditioning and Extinction: The fear conditioning protocol is identical to that previously developed and validated in the laboratory of Mohammed Milad, measuring skin conductance and Blood-Oxygen-Level-Dependent (BOLD) signal in MR images during Fear Extinction Recall. In short, a mild electric shock may be paired with two different visual stimuli and a “safe” third visually stimulus not paired with no electric shock (Conditioning phase). During a subsequent Extinction Learning phase, the subject is shown only the safe stimuli and one of the other previous visual stimuli, this time without accompanying electric shock. The subsequent Test Phase presents the subject with all three stimuli without electric shock.

Neuroimaging procedures: Neuroimaging may involve 3T non-contrast MRI and may include: 1) T1 Weighted Structural MRI; 2) Task based fMRI (Extinction recall, fearful faces, and emotional Stroop); 3) Resting state fMRI, and 4) Diffusion weighted Imaging MRI.

Genotype: DNA samples may be collected at Baseline and full genome sequencing is conducted at LGC Genomics, LLC. Analyses may be conducted in a single large batch to maximize the reliability of the genotyping.

Blood Biomarkers: Ubiquitin C-terminal Hydrolase-L1 (UCH-L1) glial fibrillary acidic protein (GFAP), neurofilament light protein (NFL), C-reactive protein (CRP), IL-6, TNF-alpha, and IL-10 levels may be assayed on Day 1 and Day 10 of the trial for all subjects.

The primary efficacy analysis may be performed on the modified intent to treat sample that includes all randomized subjects who received at least one dose of treatment and had at least one post-randomization outcome data point. To assess differences between CBD doses and placebo in change from baseline, a mixed-model repeated measures (MMRM) analysis may be used, including factors for treatment, time, and treatment-by-time interaction. The primary outcome, the TSST score at week 8 is tested two sided at the 0.05 level of significance. Secondary outcomes and contrasts may be tested sequentially in a closed testing union intersection paradigm.

### Enumerated Embodiments

E1. A method of treating an anxiety disorder in a subject in need thereof comprising administering to the subject a therapeutically effective amount of a pharmaceutical composition comprising:
at least one cannabinoid,
at least one oil;
at least one hydrophilic surfactant;
at least one co-surfactant;
less than 1 wt % water;
optionally, at least one solvent;
optionally, at least one co-solvent;
optionally, at least one phospholipid; and
optionally, at least one additive.

E2. The method of embodiment E1, wherein the anxiety disorder is social anxiety disorder, disorder generalized anxiety disorder, panic disorder, phobia-related disorder, separation anxiety disorder, selective mutism, or medication-induced anxiety disorder.

E3. The method of embodiment E2, wherein a Structured Clinical Interview Schedule for DSM-5 (SCID-5) for social anxiety disorder is reduced by the administration of the pharmaceutical composition to the subject, as compared to the subject's pre-treatment score.

E4. The method of embodiment E2, wherein a Trier Social Stress Test (TSST) score for social anxiety is reduced by the administration of the pharmaceutical composition to the subject, as compared to the subject's pre-treatment score.

E5. The method of embodiment E2, wherein a Liebowitz Social Anxiety Scale (LSAS) score for social anxiety is reduced by the administration of the pharmaceutical composition to the subject in need thereof, as compared to the subject's pre-treatment score.

E6. The method of embodiment E2, wherein a Clinical Global Impression of Severity and Improvement (CGI-I and S) score is reduced by the administration of the pharmaceutical composition to the subject in need thereof, as compared to the subject's pre-treatment score.

E7. The method of embodiment E2, wherein a General Anxiety Disorder (GAD-7) score in the subject is reduced by the administration of the pharmaceutical composition to the subject, as compared to the subject's pre-treatment score.

E8. The method of embodiment E2, wherein a Quick Inventory of Depressive Symptomology, Self-Report (QIDS-SR) score in the subject is reduced by the administration of the pharmaceutical composition to the subject, as compared to the subject's pre-treatment score.

E9. The method of embodiment E2, wherein a Perceived Stress Scale (PSS-14) score in the subject is reduced by the administration of the pharmaceutical composition to the subject, as compared to the subject's pre-treatment score.

E10. The method of embodiment E2, wherein an Anxiety Sensitivity Index-3 (ASI-3) score in the subject is reduced by the administration of the pharmaceutical composition to the subject, as compared to the subject's pre-treatment score.

E11. The method of embodiment E2, wherein an Insomnia Severity Index score in the subject is reduced by the administration of the pharmaceutical composition to the subject, as compared to the subject's pre-treatment score.

E12. The method of embodiment E2, wherein a PROMIS Anxiety score in the subject is reduced by the administration of the pharmaceutical composition to the subject, as compared to the subject's pre-treatment score.

E13. The method of embodiment E2, wherein a PROMIS Emotional Distress score in the subject is reduced by the administration of the pharmaceutical composition to the subject, as compared to the subject's pre-treatment score.

E14. The method of embodiment E2, wherein an Altman Self-Rating Mania Scale (ASRM) score in the subject is reduced by the administration of the pharmaceutical composition to the subject, as compared to the subject's pre-treatment score.

E15. The method of embodiment E2, wherein a Patient Health Questionnaire 15 Somatic Symptom Severity Scale (PHQ-15) score in the subject is reduced by the administration of the pharmaceutical composition to the subject, as compared to the subject's pre-treatment score.

E16. The method of embodiment E2, wherein a Patient Health Questionnaire 15 Somatic Symptom Severity Scale (PHQ-15) score in the subject is reduced by the administration of the pharmaceutical composition to the subject, as compared to the subject's pre-treatment score.

E17. The method of embodiment E2, wherein a PROMIS Sleep Disturbance score in the subject is reduced by the administration of the pharmaceutical composition to the subject, as compared to the subject's pre-treatment score.

E18. The method of embodiment E2, wherein a Florida Obsessive-Compulsive Inventory (FOCI) Severity Scale (Part B) score in the subject is reduced by the administration of the pharmaceutical composition to the subject, as compared to the subject's pre-treatment score.

E19. The method of embodiment E2, wherein a Substance Use score in the subject is reduced by the administration of the pharmaceutical composition to the subject, as compared to the subject's pre-treatment score.

E20. The method of embodiment E2, wherein a Patient Health Questionnaire (PHQ-9) score in the subject is reduced by the administration of the pharmaceutical composition to the subject, as compared to the subject's pre-treatment score.

E21. The method of embodiment E2, wherein a Severity Measure for Separation Anxiety Disorder score in the subject is reduced by the administration of the pharmaceutical composition to the subject, as compared to the subject's pre-treatment score.

E22. The method of embodiment E2, wherein a Severity Measure for Specific Phobia score in the subject is reduced by the administration of the pharmaceutical composition to the subject, as compared to the subject's pre-treatment score.

E23. The method of embodiment E2, wherein a Severity Measure for Agoraphobia score in the subject is reduced by the administration of the pharmaceutical composition to the subject, as compared to the subject's pre-treatment score.

E24. The method of embodiment E2, wherein a Brief Dissociative Experiences Scale (DES-B) score in the subject is reduced by the administration of the pharmaceutical composition to the subject, as compared to the subject's pre-treatment score.

E25. The method of any one of embodiments E1-E24, wherein the total daily dose of cannabinoid administered to the subject is at least 50 mg/day.

E26. The method of any one of embodiments E1-E1325, wherein the total daily dose of cannabinoid administered to the subject is from 100 mg/day to 2000 mg/day.

E27. The method of any one of embodiments E1-E26, wherein the total daily dose of cannabinoid administered to the subject is from 200 mg/day to 1400 mg/day.

E28. The method of any one of embodiments E1-E27, wherein the total daily dose of cannabinoid administered to the subject is from 200 mg/day to 600 mg/day.

E29. The method of any one of embodiments E1-E27, wherein the total daily dose of cannabinoid administered to the subject is from 700 mg/day to 1400 mg/day.

E30. The method of any one of embodiments E1-E26, wherein the total daily dose of cannabinoid administered to the subject is 200 mg/day, 400 mg/day, 600 mg/day, 700 mg/day, 1400 mg/day, or 2000 mg/day.

E31. The method of any one of embodiments E1-E25, wherein the total daily dose of cannabinoid administered to the subject is from 50 mg/day to 100 mg/day.

E32. The method of any one of embodiments E25-E31, wherein said total daily dose of cannabinoids is administered to the subject in a single daily dose.

E33. The method of any one of embodiments E25-E31, wherein said total daily dose of cannabinoids is administered to the subject as a split daily dose.

E34. The method of any one of embodiments 253-E31, wherein said total daily dose of cannabinoids is administered to the subject as a split daily dose comprising 2, 3, or 4 smaller doses.

E35. The method of embodiment E33 or embodiment E34, wherein said split daily dose comprises smaller doses having substantially equivalent cannabinoid concentration.

E36. The method of embodiment E33 or embodiment E34, wherein said split daily dose comprises smaller doses having inequivalent cannabinoid concentration.

E37. The method of any one of embodiments E1-E36, wherein the pharmaceutical composition is administered via at least one of route selected from topical, buccal, sublingual, dental, oral, otic, rectal, vaginal, endocervical, transdermal, subcutaneous, intravenous, intramuscular, transdermal, intranasal, inhalation, ocularly, gavage, or parenterally into the circulatory system of a subject.

E38. The method of embodiment E37, wherein the pharmaceutical composition is administered orally.

E39. The method of any one of embodiments E1-E36, wherein the pharmaceutical composition is administered in at least one form selected from a tablet, a gel, a powder, a lotion, an oil, a soap, a spray, an emulsion, a cream, an ointment, a capsule, soft gel capsules, chewing gum, a patch, a buccal-patch a nutraceutical a dietary supplement, or a solution.

E40. The method of embodiment E39, wherein the pharmaceutical composition is administered as a tablet, a capsule, a soft gel capsule, or a solution.

E41. The methods of any one of embodiments E1-E38, wherein the pharmaceutical composition is in the form of a soft gel capsule for administration orally as a split daily dose and provides a total daily dose of CBD of 600 mg/day to 2000 mg/day.

E42. The methods of any one of embodiments E1-E38, wherein the pharmaceutical composition is in the form of a soft gel capsule for administration orally as a split daily dose and provides a total daily dose of CBD of 400 mg/day.

E43. The methods of any one of embodiments E1-E42, wherein the total daily dose of CBD changes over the course of treatment.

E44. The methods of any one of embodiments E1-E43, wherein the total daily dose of CBD increases from 200 mg/day CBD to 400 mg/day CBD after 1 week of treatment.

E45. The methods of any one of embodiments E1-E43, wherein the total daily dose of CBD increases from 200 mg/day CBD to 400 mg/day CBD after 2 weeks of treatment.

E46. The methods of any one of embodiments E1-E43, wherein the total daily dose of CBD increases from 400 mg/day CBD to 600 mg/day CBD after 2 weeks of treatment.

E47. The method of any one of embodiments E1-E46, wherein the pharmaceutical composition comprises between 0.1 and 20 wt % of at least one cannabinoid.

E48. The method of embodiment E47, wherein the pharmaceutical composition comprises between 0.1 and 12 wt % of at least one cannabinoid.

E49. The method of embodiment E47, wherein the pharmaceutical composition comprises between 5 and 12 wt % of at least one cannabinoid.

E50. The method of embodiment E47, wherein the pharmaceutical composition comprises between 4 and 11 wt % of at least one cannabinoid.

E51. The method of embodiment E47, wherein the pharmaceutical composition comprises between 5 and 10 wt % of at least one cannabinoid.

E52. The method of any one of embodiments E1-E51, wherein the at least one cannabinoid is a non-psychoactive cannabinoid.

E53. The method of embodiment E52, wherein the non-psychoactive cannabinoid is cannabidiol (CBD).

E54. The method of embodiment E52, wherein the non-psychoactive cannabinoid is cannabigerol (CBG).

E55. The method of embodiment E52, wherein the non-psychoactive cannabinoid is cannabichromene (CBC).

E56. The method of embodiment E52, wherein the non-psychoactive cannabinoid is cannabidivarin (CBDV).

E57. The method of embodiment E52, wherein the non-psychoactive cannabinoid is a derivative of a cannabinoid selected from CBD, CBG, CBC, or CBDV.

E58. The method of any one of embodiments E1-E57, wherein the pharmaceutical composition comprises between 0.5 and 20 wt % of at least one oil.

E59. The method of embodiment E58, wherein the pharmaceutical composition comprises between 1 and 10 wt % of at least one oil.

E60. The method of embodiment E58, wherein the pharmaceutical composition comprises between 3 and 6 wt % of at least one oil.

E61. The method of any one of embodiments E1-E60, wherein the pharmaceutical composition comprises between 30 and 85 wt % of at least one hydrophilic surfactant.

E62. The method of embodiment E61, wherein the pharmaceutical composition comprises between 35 and 80 wt % of at least one hydrophilic surfactant.

E63. The method of embodiment E61, wherein the pharmaceutical composition comprises between 45 and 80 wt % of at least one hydrophilic surfactant.

E64. The method of embodiment E61, wherein the pharmaceutical composition comprises between 45 and 55 wt % of at least one hydrophilic surfactant.

E65. The method of embodiment E61, wherein the pharmaceutical composition comprises between 70 and 80 wt % of at least one hydrophilic surfactant.

E66. The method of any one of embodiments E1-E65, wherein the pharmaceutical composition comprises between 1 and 50 wt % of at least one co-surfactant.

E67. The method of embodiment E66, wherein the pharmaceutical composition comprises between 2 and 45 wt % of at least one co-surfactant.

E68. The method of embodiment E66, wherein the pharmaceutical composition comprises between 2 and 5 wt % of at least one co-surfactant.

E69. The method of any one of embodiments E1-E68, wherein the pharmaceutical composition comprises less than 1% water.

E70. The method of any one of embodiments E1-E69, wherein the pharmaceutical composition comprises between 0.1 and 25 wt % of at least one solvent.

E71. The method of embodiment E70, wherein the pharmaceutical composition comprises between 0.1 and 15 wt % of at least one solvent.

E72. The method of any one of embodiments E1-E71, wherein the pharmaceutical composition comprises at least one co-solvent.

E73. The method of any one of embodiments E1-E72, wherein the pharmaceutical composition comprises between 1 and 10 wt % of at least one phospholipid.

E74. The method of any one of embodiments E1-E73, wherein the pharmaceutical composition comprises between 0.1 and 10 wt % of at least one additive.

E75. The method of claim E74, wherein the pharmaceutical composition comprises between 5 and 7 wt % of at least one additive.

E76. The method of claim E74, wherein the pharmaceutical composition comprises between 8 and 10 wt % of at least one additive.

E77. A kit comprising the pharmaceutical composition of any one of embodiments E1-E76 and instructions for the use of said composition.

E78. A kit for the treatment of anxiety disorder, said kit comprising a pharmaceutical composition and instructions for carrying out the method of any one of embodiments E1-E76.

E79. A method of producing a Probability of Response (POR) of a subject to any of the methods of treating anxiety disorder recited in the preceding claims, said method essentially comprising:
collecting a subject's demographic, clinical (including medication and substance use information), and biological (i.e., genetic, and other blood-based markers) information as well as task-based and structural magnetic resonance neuroimaging markers centered around hippocampal, prefrontal, and extended amygdala function and fear extinction before administration of the pharmaceutical composition or placebo; and
administering the pharmaceutical composition of any one of embodiments E1-E76 or placebo and collecting the measures of again after administration; and applying a random forest algorithm to generate a POR.

E80. A method for selecting subjects most likely to benefit from any of the methods of treating anxiety disorder recited in the preceding claims, said method essentially comprising:
collecting a subject's demographic, clinical (including medication and substance use information), and biological (i.e., genetic, and other blood-based markers) information as well as task-based and structural magnetic resonance neuroimaging markers centered around hippocampal, prefrontal, and extended amygdala function and fear extinction;
applying a probability classifier to the features collected to predict the POR to the pharmaceutical composition of any one of embodiments E1-E76 for the subject; and
selecting subjects who have a predicted POR greater than a determined threshold (e.g., POR >0.6).

### OTHER EMBODIMENTS

While the invention has been described in connection with specific embodiments thereof, it will be understood that it is capable of further modifications and this application is intended to cover any variations, uses, or adaptations of the invention following, in general, the principles of the disclosure and including such departures from the invention that come within known or customary practice within the art to which the disclosure pertains and may be applied to the essential features hereinbefore set forth, and follows in the scope of the claims. Other embodiments are within the claims.

1-50. (canceled)

51. A method of treating an anxiety disorder in a subject in need thereof comprising administering to the subject a therapeutically effective amount of a pharmaceutical composition comprising:
at least one cannabinoid,
at least one oil;
at least one hydrophilic surfactant;
at least one co-surfactant;
less than 1 wt % water;
optionally, at least one solvent;
optionally, at least one co-solvent;
optionally, at least one phospholipid; and
optionally, at least one additive.

52. The method of claim 51, wherein the anxiety disorder is selected from the group consisting of social anxiety disorder, disorder generalized anxiety disorder, panic disorder, phobia-related disorder, separation anxiety disorder, selective mutism, and medication-induced anxiety disorder.

53. The method of claim 52, wherein administration of the pharmaceutical composition to the subject causes reduction in one or more scores as compared to the subject's pre-administration score, said score being selected from the group consisting of: a Structured Clinical Interview Schedule for DSM-5 (SCID-5) score for social anxiety disorder, a Trier Social Stress Test (TSST) score for social anxiety, a Liebowitz Social Anxiety Scale (LSAS) score for social anxiety, a Clinical Global Impression of Severity and Improvement (CGI-I and S) score, a General Anxiety Disorder (GAD-7) score, a Quick Inventory of Depressive Symptomology Self-Report (QIDS-SR) score, a Perceived Stress Scale (PSS-14) score, an Anxiety Sensitivity Index-3 (ASI-3) score, an Insomnia Severity Index score, a PROMIS Anxiety score, a PROMIS Emotional Distress score, an Altman Self-Rating Mania Scale (ASRM) score, a Patient Health Questionnaire 15 Somatic Symptom Severity Scale (PHQ-15) score, a PROMIS Sleep Disturbance score, a Florida Obsessive-Compulsive Inventory (FOCI) Severity Scale (Part B) score, a Substance Use score, a Patient Health Questionnaire (PHQ-9) score, a Severity Measure for Separation Anxiety Disorder score, a Severity Measure for Specific Phobia score, a Severity Measure for Agoraphobia score, and a Brief Dissociative Experiences Scale (DES-B) score.

54. The method of claim 51, wherein the total daily dose of cannabinoid administered to the subject is at least 50 mg/day.

55. The method of claim 51, wherein the total daily dose of cannabinoid administered to the subject is from 100 mg/day to 2000 mg/day.

56. The method of claim 51, wherein the total daily dose of cannabinoid administered to the subject is from 200 mg/day to 1400 mg/day.

57. The method of claim 56, wherein the total daily dose of cannabinoid administered to the subject is from 200 mg/day to 600 mg/day.

58. The method of claim 56, wherein the total daily dose of cannabinoid administered to the subject is from 700 mg/day to 1400 mg/day.

59. The method of claim 54, wherein said total daily dose of cannabinoids is administered to the subject in a single daily dose.

60. The method of claim 54, wherein said total daily dose of cannabinoids is administered to the subject as a split daily dose.

61. The method of claim 60, wherein said split daily dose comprises smaller doses having substantially equivalent cannabinoid concentration.

62. The method of claim 60, wherein said split daily dose comprises smaller doses having inequivalent cannabinoid concentration.

63. The method of claim 51, wherein the pharmaceutical composition is administered via at least one of route selected from the group consisting of topical, buccal, sublingual, dental, oral, otic, rectal, vaginal, endocervical, transdermal, subcutaneous, intravenous, intramuscular, transdermal, intranasal, inhalation, ocularly, gavage, and parenterally into the circulatory system of a subject.

64. The method of claim 63, wherein the pharmaceutical composition is administered orally.

65. The method of claim 64, wherein the pharmaceutical composition is administered as a tablet, a capsule, a soft gel capsule, or a solution.

66. The method of claim 51, wherein the pharmaceutical composition is in the form of a soft gel capsule for administration orally as a split daily dose and provides a total daily dose of CBD of 400 mg/day to 2000 mg/day.

67. The method of claim 51, wherein the total daily dose of CBD changes over the course of treatment.

68. The method of claim 67, wherein the total daily dose of CBD increases from 200 mg/day CBD to 400 mg/day CBD after 1 week of treatment.

69. The method of claim 67, wherein the total daily dose of CBD increases from 200 mg/day CBD to 400 mg/day CBD after 2 weeks of treatment.

70. The method of claim 67, wherein the total daily dose of CBD increases from 400 mg/day CBD to 600 mg/day CBD after 2 weeks of treatment.
